(19) United States
(12) Patent Application Publication
(10) Pub. No.: US 2025/0154514 A1
(43) Pub. Date: May 15, 2025

(54) THERMOPHILIC SITE-SPECIFIC RECOMBINATION SYSTEM FOR RAPID INSERTION OF HETEROLOGOUS DNA

(71) Applicant: UT-Battelle, LLC, Oak Ridge, TN (US)

(72) Inventors: Adam M. Guss, Knoxville, TN (US); Nandhini Ashok, Knoxville, TN (US); Heidi S. Schindel, Knoxville, TN (US)

(21) Appl. No.: 18/947,263

(22) Filed: Nov. 14, 2024

Related U.S. Application Data
(60) Provisional application No. 63/599,027, filed on Nov. 15, 2023.

(51) Int. Cl. C12N 15/74; C12N 9/52

(52) U.S. Cl. CPC C12N 15/74; C12N 9/52

(57) ABSTRACT

The current disclosure relates to a genetically engineered thermophile bacterial cell comprising at least one att site, a system for stable insertion of a heterologous DNA into a thermophile bacterial cell, a method for the thermostable insertion of a heterologous DNA into a chromosome of an organism, and a thermophile bacterial cell made through the methods disclosed. The disclosure is also directed to a thermophile bacterial cell, comprising a cargo plasmid comprising a heterologous DNA inserted in the chromosome of the bacterial cell, wherein the cargo plasmid is flanked by an attL site and an attR site. The disclosure is directed to a thermophile bacterial cell, comprising in its chromosome, a DNA flanked by a pair of attB and attP recombination sites. Lastly, the disclosure relates to a system for excising DNA from the chromosome of an organism and a method for excising DNA from the chromosome of an organism.

### CROSS REFERENCE TO RELATED APPLICATIONS

This application claims the benefit of U.S. Provisional Patent Application No. 63/599,027, filed Nov. 15, 2023, the contents of which is incorporated herein by reference in its entirety.

### STATEMENT REGARDING FEDERALLY SPONSORED RESEARCH

The United States Government has rights in this invention pursuant to contract no. DE-AC05-00OR22725 between the United States Department of Energy and UT-Battelle, LLC.

### INCORPORATION BY REFERENCE OF SEQUENCE LISTING

The Sequence Listing in a XML file, named as 43615_4773_1_SequenceListing.xml of 52,000 bytes, created on Nov. 14, 2024, and submitted to the United States Patent and Trademark Office via Patent Center, is incorporated herein by reference.

### BACKGROUND

Clostridium thermocellum is an anaerobic thermophile capable of metabolizing complex and heterogenous lignocellulosic biomass. Its native metabolic pathways produce various commodity chemicals such as fermentation products, including ethanol, acetate, lactate, formate, H2, isobutanol, 2,3 butanediol, and free amino acids. Research in the past decade has focused on metabolic engineering of C. thermocellum to make industrially significant amounts of a specific fuel or commodity chemical, primarily ethanol. One of the bottlenecks in exploring desirable genetic modifications with this organism is the limited throughput allowed by its few and time-consuming genetic tools.

Because C. thermocellum transformation efficiency is too low to directly select for homologous recombination of a non-replicating plasmid into the chromosome during transformation, stable insertion of heterologous DNA into the C. thermocellum chromosome is a multi-step process that utilizes a temperature-sensitive replicating plasmid based on pNW33N. One option utilizes three regions of homology, as well as one selectable and two counter-selectable markers. Alternatively, a temperature-sensitive replicating plasmid with the gene(s) of interest flanked between homologous arms can be transformed into C. thermocellum selecting thiamphenicol resistance encoded by the cat gene, followed by incubation at a non-permissive temperature (60° C.) to select for genomic integration via homologous recombination. Finally, a counter-selectable marker gene like hpt or tdk present on the plasmid (now genomically integrated) allows selection for the second recombination event in the presence of corresponding anti-metabolites (8-azahypoxanthine and 5-fluoro-2′-deoxyuridine, respectively). This protocol typically takes weeks to complete in wild type C. thermocellum and even longer in slower-growing mutant strains, with each final colony having a 50% chance of being the strain of interest in the final step. It is thus a significant bottleneck for screening chromosomally integrated genetic parts (e.g., promoters, reporters, ribosome binding sites) and heterologous pathways for metabolic engineering.

Serine recombinase Assisted Genome Engineering (SAGE) is a method that has been recently developed for rapid and simple genomic integration of genetic cassettes into model and non-model organisms like Escherichia coli, Pseudomonas sp., Rhodococcus jostii, and Rhodopseudomonas palustris. SAGE uses a large serine recombinase to facilitate a site-specific recombination event between two non-identical base pair DNA sequences, called attB and attP sites. Recombination between these attB and attP sites, collectively called att sites, results in the formation of new attL and attR sites, leaving genetic “scars” that are not substrates for further recombination, making the recombination reaction irreversible and stable. This is unlike the FLP-frt and the CRE-lox tyrosine recombinase-mediated systems, which are reversible and can result in strain instability issues when used for genome engineering. SAGE has been shown to work in mesophilic organisms, but not yet in thermophilic organisms.

### SUMMARY

In one aspect, the present disclosure is directed to a genetically engineered thermophile bacterial cell comprising at least one att site. In another aspect, the disclosure is directed to a system for stable insertion of a heterologous DNA into a thermophile bacterial cell. In a further aspect, the disclosure is directed to a method for the thermostable insertion of a heterologous DNA into a chromosome of an organism. In one aspect, the disclosure is directed to a thermophile bacterial cell made through the methods disclosed in the disclosure. In another aspect, the disclosure is directed to a thermophile bacterial cell, comprising a cargo plasmid comprising a heterologous DNA inserted in the chromosome of the bacterial cell, wherein the cargo plasmid is flanked by an attL site and an attR site. In a further aspect, the disclosure is directed to a thermophile bacterial cell, comprising in its chromosome, a DNA flanked by a pair of attB and attP recombination sites. In one aspect, the disclosure is directed to a system for excising DNA from the chromosome of an organism. In another aspect, the disclosure is directed to a method for excising DNA from the chromosome of an organism.

In one aspect, the present disclosure is directed to a genetically engineered thermophile bacterial cell comprising at least one att site in its chromosome wherein the att site is one member of a pair of attB and attP recombination sites. In some embodiments, the cell expresses a thermophilic site-specific recombinase that recognizes the pair of attB and attP recombination sites. In some embodiments, the at least one att site comprises multiple att sites, each being a member of a pair of attB and attP recombination sites recognized by different site-specific recombinases.

In another aspect, the present disclosure is directed to a system for stable insertion of a heterologous DNA, the system comprising:
a thermophile bacterial cell comprising at least one att site in its chromosome wherein the att site is one member of a pair of attB and attP recombination sites; and
a cargo plasmid comprising the heterologous DNA and a corresponding att site, wherein the corresponding att site is the other member of the pair of attB and attP recombination sites;
wherein the cargo plasmid is capable of insertion into the chromosome of the thermophile at the at least one att site via recombination with the corresponding att site on the cargo plasmid; and
wherein the recombination is mediated by a thermophilic site-specific recombinase that recognizes the pair of attB and attP recombination sites, and results in the formation of an attL site and an attR site, wherein the attL and attR sites are not substrates for further recombination.

In some embodiments, the thermophile bacterial cell comprises a native att site. In some embodiments the thermophile bacterial cell comprises a genetically engineered att site. In some embodiments, the thermophile bacterial cell comprises both native att and genetically engineered att sites. In some embodiments, the thermophile bacterial cell expresses the thermophilic site-specific recombinase. In some embodiments, the disclosed system further comprises a nucleic acid encoding the thermophilic site-specific recombinase. In some embodiments, the nucleic acid encoding the thermophilic site-specific recombinase is provided on a helper plasmid. In some embodiments, the cargo plasmid further comprises a selectable marker gene. In some embodiments, the selectable marker gene is flanked by another pair of pair of attB and attP recombination sites recognized by another thermophilic site-specific recombinase. In some embodiments, the thermophilic site-specific recombinase and the another thermophilic site-specific recombinase are a serine recombinase or a tyrosine recombinase. In some embodiments, the serine recombinase is selected from Y412MC61, BXB1, and TG1, and homologs thereof. In some embodiments, the nucleic acid encoding the serine recombinase is a nucleic acid encoding the Y412MC61 recombinase comprising a nucleotide sequence of SEQ ID NO: 1; the nucleic acid encoding the serine recombinase is a nucleic acid encoding a Y412MC61 recombinase homolog comprising a nucleotide sequence having at least 90% sequence identity to the nucleotide sequence shown in SEQ ID NO: 1; the nucleic acid encoding the serine recombinase is a nucleic acid encoding the BXB1 recombinase comprising a nucleotide sequence of SEQ ID NO: 3; the nucleic acid encoding the serine recombinase is a nucleic acid encoding a BXB1 recombinase homolog comprising a nucleotide sequence having at least 90% sequence identity to SEQ ID NO: 3; the nucleic acid encoding the serine recombinase is a nucleic acid encoding the TG1 recombinase comprising a nucleotide sequence of SEQ ID NO: 5; or the nucleic acid encoding the serine recombinase is a nucleic acid encoding a TG1 recombinase homolog comprising a nucleotide sequence having at least 90% sequence identity to SEQ ID NO: 5.

In some embodiments, the Y412MC61 recombinase comprises an amino acid sequence of SEQ ID NO: 2; aY412MC61 recombinase homolog comprises an amino acid sequence having at least 90% sequence identity to the amino acid sequence shown in SEQ ID NO: 2; the BXB1 recombinase comprises an amino acid sequence of SEQ ID NO: 4; a BXB1 recombinase homolog comprises an amino acid sequence having at least 90% sequence identity to the amino acid sequence shown in SEQ ID NO: 4; the TG1 recombinase comprises an amino acid sequence of SEQ ID NO: 6; or a TG1 recombinase homolog comprises an amino acid sequence having at least 90% sequence identity to the amino acid sequence shown in SEQ ID NO: 6.

In some embodiments, the thermophile bacterium cell is from the genus of Bacillus, Geobacillus, Paenebacillus, Clostridium, Anaerocellum, Caldicellulosiruptor, Thermus, Pyrococcus, Thermococcus, Thermoanaerobacter, Thermoanaerobacterium, Herbinix, Acetivibrio, Acidothermus. In some embodiments, the bacterium cell is a Clostridium thermocellum, Geobacillus thermoglucosidasius, or Bacillus licheniformis.

In some embodiments, the thermophile bacterial cell comprises multiple att sites in its chromosome, and the system comprises another cargo plasmid comprising another corresponding att site.

In another aspect, the disclosure is directed to a method for the thermostable insertion of a heterologous DNA into a chromosome of an organism, the method comprising:
a) transfecting a cargo plasmid into a thermophile bacterial cell comprising at least one att site in its chromosome wherein the att site is one member of a pair of attB and attP recombination sites,
wherein the thermophile bacterial cell expresses a thermophilic site-specific recombinase that recognizes the pair of attB and attP recombination sites;
wherein the cargo plasmid comprising a corresponding att recombination site, wherein the corresponding att recombination site is the other member of the pair of attB and attP recombination sites;
wherein the cargo plasmid comprises the heterologous DNA;
wherein the cargo plasmid is capable of insertion into the chromosome of the thermophile bacterial cell at the at least one att site via recombination with the at least one corresponding att site on the cargo plasmid; and
wherein the recombination results in the formation of an attL site and an attR site, wherein the attL and attR sites are not substrates for further recombination.
wherein the insertion is a site-specific recombination at a temperature at or above 50° C. and is thermostable; and
b) selecting a thermophile bacterial cell wherein the heterologous DNA is inserted into the chromosome of the thermophile bacterial cell.

In some embodiments, the thermophile bacterial cell comprises a native att site. In some embodiments the thermophile bacterial cell comprises a genetically engineered att site. In some embodiments, the thermophile bacterial cell comprises both native att and genetically engineered att sites. In some embodiments, the expression of the thermophilic site-specific recombinase is achieved by transfecting, in step (a), a helper plasmid into the thermophile bacterial cell, wherein the helper plasmid comprises a nucleic acid sequence encoding the site-specific recombinase. In some embodiments, the method further comprises c) culturing the selected bacterial cell under conditions suitable for growth and replication. In some embodiments, the culturing comprises culturing at a temperature at or above 55° C. In some embodiments, the cargo plasmid further comprises a selectable marker gene. In some embodiments, the selecting in step (b) is based on the selectable marker. In some embodiments, the selectable marker gene is flanked by another pair of attB and attP recombination sites recognized by another thermophilic site-specific recombinase. In some embodiments, the selectable marker gene is removed from the selected thermophile bacterial cell via recombination mediated by the another thermophilic site-specific recombinase. In some embodiments, the another thermophilic site-specific recombinase is expressed from a helper plasmid introduced into the selected thermophile bacterial cell. In some embodiments, the thermophilic site-specific recombinase and the another thermophilic site-specific recombinase are a serine recombinase or a tyrosine recombinase. In some embodiments, the serine recombinase is Y412MC61, BXB1 or TG1.

In some embodiments of the method for the thermostable insertion of a heterologous DNA into a chromosome of an organism, the thermophile bacterial cell comprises multiple att sites in its chromosome, wherein the method comprises transfecting into the thermophile bacterial cell another cargo plasmid comprising another corresponding att site and another heterologous DNA, and selecting a thermophile bacterial cell in which the another heterologous DNA is also integrated in the chromosome.

In one aspect, the disclosure is directed to a thermophile bacterial cell made through the method for the thermostable insertion of a heterologous DNA into a chromosome of an organism as disclosed herein.

In another aspect, the disclosure is directed to a thermophile bacterial cell, comprising a cargo plasmid comprising a heterologous DNA inserted in the chromosome of the bacterial cell, wherein the cargo plasmid is flanked by an attL site and an attR site.

In still another aspect, the disclosure is directed to a thermophile bacterial cell, comprising in its chromosome, a DNA flanked by a pair of attB and attP recombination sites. In some embodiments, the DNA is a DNA native to the thermophile bacterial cell. In some embodiments, the DNA is a DNA heterologous to the thermophile bacterial cell.

In another aspect, the disclosure is directed to a system for excising DNA from the chromosome of an organism, the system comprising:
the thermophile bacterial cell comprising in its chromosome, a DNA flanked by a pair of attB and attP recombination sites; and
a nucleic acid encoding a site-specific recombinase that performs recombination between the attB and attP recombination sites; and
wherein the recombination results in the formation of an attL site and an attR site, wherein the attL and attR sites are not substrates for further recombination, and the DNA is removed from the chromosome.

In some embodiments of the disclosure, the thermophilic site-specific recombinase is a serine recombinase or a tyrosine recombinase. In some embodiments, the serine recombinase is selected from Y412MC61, BXB1, and TG1, and homologs thereof. In some embodiments, the excised DNA is a DNA native to the thermophile bacterial cell. In some embodiments, the excised DNA is a DNA heterologous to the thermophile bacterial cell.

In one aspect, the disclosure is directed to a method for excising DNA from the chromosome of an organism, the method comprising:
providing the thermophile bacterial cell, comprising in its chromosome, a DNA flanked by a pair of attB and attP recombination site; and
expressing a thermophilic site-specific recombinase that performs recombination between the attB and attP recombination sites; and
wherein the recombination results in the formation of an attL site and an attR site, wherein the attL and attR sites are not substrates for further recombination, and the DNA is removed from the chromosome.

In some embodiments, the thermophilic site-specific recombinase is expressed from a plasmid introduced into the thermophile bacterial cell. In some embodiments, the thermophilic site-specific recombinase is a serine recombinase or a tyrosine recombinase. In some embodiments, the serine recombinase is selected from Y412MC61, BXB1, and TG1, and homologs thereof. In some embodiments, the excised DNA is a DNA native to the thermophile bacterial cell. In some embodiments, the excised DNA is a DNA heterologous to the thermophile bacterial cell.

In another aspect, the disclosure is directed to a thermophile bacterial cell, comprising in its chromosome, a DNA flanked by a pair of attB and attP recombination sites. In some embodiments, the DNA is a DNA native to the thermophile bacterial cell. In some embodiments, the DNA is a DNA heterologous to the thermophile bacterial cell.

In still another aspect, the disclosure is directed to a thermophile bacterial cell made through the method for excising DNA from the chromosome of an organism disclosed herein.

### BRIEF DESCRIPTION OF DRAWINGS

FIG. 1A-D. Thermostable Serine recombinase Assisted Genome Engineering (tSAGE) overview. A) A markerless and scarless parent strain with a genomically integrated Geobacillus Y412MC61 attB site is constructed using homologous recombination described elsewhere [PMID: 21965408]. This parent strain is used for all tSAGE integrations. B) The integrating cargo plasmid contains a chloramphenicol acetyl transferase (catR) gene, the genetic cargo of interest and a Geobacillus Y412MC61 attP site. The helper plasmid expressed the Geobacillus Y412MC61 serine recombinase. The cargo and the helper plasmids are co-transformed into competent cells of the parent strain using electroporation. C) An overnight anaerobic recovery is done at 50° C. in non-selective rich media to allow time for recombination and expression of the selectable marker. D) The recovered cells are plated in selective media and incubated anaerobically at 50° C., resulting in a strain in which the cargo and the marker are integrated into the chromosome and flanked by attL and attR sites.

FIG. 2A-C. Efficiency of cargo integration by tSAGE in C. thermocellum. A) Transformation efficiency of tSAGE integrated plasmids, reported as colony forming units (cfu) per microgram of DNA. pAMG216 is a replicating plasmid in C. thermocellum and is used as a positive control. pNA35, pHS96, pNA83 and pNA86 are non-replicating plasmids with a Y412MC61 attP site. These plasmids were integrated into the chromosome of C. thermocellum using tSAGE. B) PCRs confirming plasmid integration using the Y412MC61 serine recombinase. “L” indicates the DNA ladder. Lanes 1 through 6 indicate individual colonies tested for chromosomal plasmid integration. “P” indicates the parent strain, AG8235, with the poly-attB landing pad. PCR #1 used primers #13 and #10. Genomic integration of pHS96 at the clo1313_2366 locus results in a 1607 base pair band (lanes 1 through 6) with PCR #1. PCR #2 used primer #14 and primer #11. Genomic integration of pHS96 at the clo1313_2366 locus results in a 285 base pair band (lanes 1 through 6) with PCR2. No band is expected from either PCR in the parent strain. C) PCRs confirming plasmid integration into C. thermocellum using BXB1 and TG1 recombinases. All the PCRs have used primers that flank the clo1313_2366 locus (primer #13 and primer #14). “L” indicates the DNA ladder. A 5923 base pair band is expected when pHS96 integrates into AG8235 using BXB1 recombinase (Lanes 1 through 4). A 5923 base pair band is expected when pHS96 integrates into AG8235 using TG1 recombinase (Lanes 5 through 8). The primers result in a 1719 base pair band in the parent strain (AG8235) with the poly-attB landing pad (lane 9).

FIG. 3. Testing reporter genes. tSAGE was used to integrate a library of reporter genes into the chromosome of C. thermocellum. The strains were then assayed for fluorescence. FBFPs refers to Flavin Based Fluorescent Proteins that do not need oxygen to render fluorescence. mrFBFP is from Meiothermus ruber. CagFBFP-V1 and CagFBFP-V2 are from Chloroflexus aggregans and the YNP3FBFPs are from the YNP metagenome project. sf-GFP and mKATE are fluorescent proteins that need oxygen for the chromophore to mature. sf-GFP refers to the superfolder Green fluorescent protein and mKATE is a red fluorescent protein.

FIG. 4. Testing promoters. tSAGE was used to integrate a library of promoters driving superfolder GFP into the chromosome of C. thermocellum. The strains were then assayed for fluorescence. The promoters were from homologous (grey) and heterologous (white) sources. Promoters from C. thermocellum (clo1313), Thermoanaerobacterium saccharolyticum (Tsac), Geobacillus thermodentrificans (GTNG), Caldicellulosiruptor bescii (Athe), Acetovibrio clariflvus (Clocl), Geobacillus thermoglucosidasius (Geoth), and Streptococcus pneumoniae (SPH) were tested for expression in Clostridium thermocellum.

FIG. 5A-D. Testing regulated expression. A) Normalized fluorescence of strains with a chromosomally integrated xylose-inducible promoter from Caldicellulosiroptor bescii driving sfGFP at sequentially increasing concentrations of xylose. B) pbuE riboswitches driven by P_{clo1313_1194} and expressing sfGFP at sequentially increasing concentrations of 2-aminopurine (2-AP). C) pbuE riboswitches driven by P_{Tsac_0068} and expressing sfGFP at sequentially increasing concentrations of 2-AP. D) pbuE riboswitches driven by P_{Clo1313_gapDH} and expressing sfGFP at sequentially increasing concentrations of 2-AP.

FIG. 6. Testing different spacers between the RBS and start codon. tSAGE was used to integrate an RBS spacer library driving superfolder GFP (sfGFP) into the chromosome of C. thermocellum. The strains were then assayed for fluorescence. Black bar indicates the fluorescence of the native P1194 promoter driving sfGFP. The distance between the RBS and the start codon (ATG) were either decreased (white-above black bar) or increased (white-below black bar) relative to the native spacing of 8 bases. The spacer of each construct is underlined. The grey bar indicates fluorescence when the native RBS was modified to match the consensus RBS.

FIG. 7. PCRs confirming plasmid backbone excision using BXB1 serine recombinase. “L” indicates the DNA ladder. PCR #3 for lanes 1 through 9 have used primers that flank the clo1313_2366 locus (primer #13 and primer #14). A 2721 base pair band is expected in a Clostridium thermocellum strain without the Y412MC61 attB landing pad (lane 1), a 452 base pair band is expected in the strain with the Y412MC61 landing pad at the clo1313_2366 locus (lane 2), 4298 base pair band is expected when the plasmid-pNA122-is integrated at the Y412MC61 attB site (lane 3), and a 1799 base pair band is expected when the backbone of the plasmid pNA122 is excised at the BXB1 attP and attB sites (lanes 4 through 9). PCR #4 for lanes 10 through 18 have used primer #49 and primer #50 to check for the expression vector pNA56G. No PCR bands are expected in the C. thermocellum strain without the Y412MC61 attB landing pad (lane 10), the C. thermocellum strain the Y412MC61 landing pad (lane 11), and the C. thermocellum strain in which pNA122 has been integrated (lane 12). The backbone excised colonies all show the presence of the BXB1 expression vector with the 1843 base pair band (lanes 13 through 18).

### DETAILED DESCRIPTION

A key feature of the present disclosure is utilization of a thermophilic site-specific recombinase with a pair of attB and attP sites that are uniquely recognized by the thermophilic site-specific recombinase to achieve insertion of a DNA into the chromosome of a thermophilic bacterial cell. For example, one member of the attB and attP pair is placed on the chromosome of the thermophile while the other member of the pair, along with the DNA to be inserted, is included on a cargo plasmid. The site-specific recombinase is expressed in the thermophile. The expression of the site-specific recombinase can be accomplished via a helper plasmid which is also introduced into the thermophile. Introduction of the cargo plasmid into in the thermophile, combined with the expression of the site-specific recombinase in the thermophile, leads to the insertion of the DNA into the chromosome. Similarly, the thermophilic site-specific recombinase works with a pair of attB and attP sites to also achieve the removal of a DNA from the chromosome of the thermophilic bacterial cell. For removal of a DNA from the chromosome, the attB and attP pair can be placed to flank the DNA. The DNA is excised upon expression of a site-specific recombinase in the thermophilic bacterial cell that recognizes the attB and attP pair. The expression of the site-specific recombinase can also be accomplished via a helper plasmid introduced into the thermophilic bacterial cell.

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, are herein incorporated by reference in their entireties.

As used herein, “bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

The terms “genetically modified,” “recombinant cell,” and “recombinant strain” are used interchangeably herein and refer to bacterial cells that have been genetically modified by the cloning and transformation methods of the present disclosure. Thus, the terms include a prokaryote that has been genetically altered, modified, or engineered, such that it exhibits an altered, modified, or different genotype and/or phenotype (e.g., when the genetic modification affects coding nucleic acid sequences of the microorganism), as compared to the naturally occurring microorganism from which it was derived. It is understood that the terms refer not only to the particular recombinant microorganism in question, but also to the progeny or potential progeny of such a microorganism.

The term “genetically engineered” may refer to any manipulation of a cell's genome (e.g. by insertion or deletion of nucleic acids).

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure, homologous sequences are compared. “Homologous sequences”, “homologs”, or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Examples of alignment programs include but are not limited to: Mac Vector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, “promoter” refers to a DNA sequence capable of controlling the expression of a coding sequence or functional RNA. The promoter sequence may consist of proximal and more distal upstream elements, the latter elements often referred to as enhancers.

As used herein, the term “heterologous” refers to a nucleic acid sequence, which is not naturally found in the particular organism.

As used herein, the term “exogenous” is used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system. Artificially mutated variants of endogenous genes are considered “exogenous” for the purposes of this disclosure.

The term “operably linked” means in this context the sequential arrangement of the promoter polynucleotide according to the disclosure with a further oligo- or polynucleotide, resulting in transcription of the further polynucleotide. In some embodiments, the promoter sequences of the present disclosure are inserted just prior to a gene's 5′UTR, or open reading frame. In other embodiments, the operably linked promoter sequences and gene sequences of the present disclosure are separated by one or more linker nucleotides.

### Site-Specific Recombination

Site-specific recombination is an exchange between two defined sites resulting in integration, excision, or inversion. Site-specific recombination makes use of enzymes (recombinases, transposases, integrases), which catalyze DNA strand exchange between DNA molecules that have only limited sequence homology. DNA cleavage at the recombination site results in an intermediate with the recombinase covalently linked to the ends of the DNA; reversal of this process reseals the DNA to form the recombinant and releases the recombinase.

As used herein, “site-specific recombinases” refer to enzymes that catalyze site-specific recombination between two specific DNA sequences to mediate DNA integration, excision, resolution, or inversion. Site-specific recombinases play a pivotal role in the life cycles of many microorganisms including bacteria and bacteriophages. There are two classifications of site-specific recombinases based on whether a tyrosine or serine residue mediates catalysis, tyrosine-type and serine-type recombinases. The most well-known site-specific recombinases are Cre, Flp and ΦC31 integrase. Cre and Flp each recognize their own recombination sites, lox sites for Cre and frt sites for Flp. The sites resulting from recombination mediated by Cre and Flp are still substrates for additional recombination, i.e. a recombination using Cre or Flp recombinase is reversible.

As used herein, “thermophilic recombinases” are site-specific recombinases that can be used at thermophilic temperatures, i.e. site-specific recombinases that work in thermophilic organisms. Therefore, thermophilic recombinases are site-specific recombinases that are thermostable. Thermophilic temperatures are temperatures at or above 50° C. where thermophiles can survive or thrive. In some embodiments, thermophilic recombinases are site-specific recombinases that come from thermophile bacteria. For example, a previous group computationally predicted some serine recombinases and their cognate attP and attB sites in various prokaryotes, including one from Geobacillus sp. Y412MC61. Geobacillus strains are able to grow at thermophilic temperatures, suggesting the Y412MC61 recombinase might be thermostable. As such, SAGE might be adaptable to thermophilic organisms through the use of the Y412MC61 recombinase. Another Geobacillus recombinase from Parageobacillus thermoglucosidasius C56-YS93, which is also called Geobacillus thermoglucosidasius, has also been used in Geobacillus thermoglucosidasius using a native attB site, further suggesting that it could be adapted to other thermophilic organisms like C. thermocellum.

As used herein, “recombination site” or “recognition site” refers to the nucleotide sequence specifically recognized by a site-specific recombinase and it is also the location where the recombination of the DNA occurs. Recombination sites are typically between 30 and 200 nucleotides in length and comprise two motifs with a partial inverted-repeat symmetry, to which the recombinase binds. The two motifs flank a central crossover sequence at which the recombination takes place. Recognition sites of site-specific recombinases (e.g. Cre, Flp, or ΦC31 recombinase) are usually around 30-100 base pair DNA sequences. In some embodiments, the recombination site is referred to as an attB site and an attP site.

The systems of the present disclosure involve pairs of recombination sites. A “pair” of recombination sites are cognate recombination sites which work together for the recombination to take place. The cognate recombination sites are sites of different sequences, but each site is necessary for the implementation of the DNA into the chromosome. One member of a pair of recombination sites is referred to as an “att” site herein. When referred to as an “att” site, this means that the first member of the pair of recombination sites can be either of the pair of recombination sites, while the second member of the pair of recombination sites is the cognate, or other, member of the pair of recombination sites. In some embodiments, one member of the pair of recombination sites is an attB site while the cognate member of the pair of recombination sites is an attP recombination site, or vice versa. In some embodiments, the attB site is located in the bacterial genome while the attP site is located in the cargo plasmid. In some embodiments, the attB site is located in the cargo plasmid while the attP site is located in the bacterial genome. In some embodiments, the recombination site of the bacterial genome is native to the genome, i.e. the bacterial cell naturally comprises the recombination site in its genome. In some embodiments, the recombination site of the bacterial genome is genetically engineered into the bacterial genome.

In some embodiments, the pair of attB/attP recombination sites is specific to a Y412MC61 recombinase. In some embodiments, the attB and attP recombination sites each have a nucleotide sequence specific to a Y412MC61 recombinase. In some embodiments, the Y412MC61 attB site has a nucleotide sequence that is at least 90% identical to SEQ ID NO: 7 and the Y412MC61 attP site has a nucleotide sequence that is at least 90% identical to SEQ ID NO: 8. In some embodiments, the Y412MC61 attB site has a nucleotide sequence that is at least 91% identical to SEQ ID NO: 7 and the Y412MC61 attP site has a nucleotide sequence that is at least 91% identical to SEQ ID NO: 8. In some embodiments, the Y412MC61 attB site has a nucleotide sequence that is at least 92% identical to SEQ ID NO: 7 and the Y412MC61 attP site has a nucleotide sequence that is at least 92% identical to SEQ ID NO: 8. In some embodiments, the Y412MC61 attB site has a nucleotide sequence that is at least 93% identical to SEQ ID NO: 7 and the Y412MC61 attP site has a nucleotide sequence that is at least 93% identical to SEQ ID NO: 8. In some embodiments, the Y412MC61 attB site has a nucleotide sequence that is at least 94% identical to SEQ ID NO: 7 and the Y412MC61 attP site has a nucleotide sequence that is at least 94% identical to SEQ ID NO: 8. In some embodiments, the Y412MC61 attB site has a nucleotide sequence that is at least 95% identical to SEQ ID NO: 7 and the Y412MC61 attP site has a nucleotide sequence that is at least 95% identical to SEQ ID NO: 8. In some embodiments, the Y412MC61 attB site has a nucleotide sequence that is at least 96% identical to SEQ ID NO: 7 and the Y412MC61 attP site has a nucleotide sequence that is at least 96% identical to SEQ ID NO: 8. In some embodiments, the Y412MC61 attB site has a nucleotide sequence that is at least 97% identical to SEQ ID NO: 7 and the Y412MC61 attP site has a nucleotide sequence that is at least 97% identical to SEQ ID NO: 8. In some embodiments, the Y412MC61 attB site has a nucleotide sequence that is at least 98% identical to SEQ ID NO: 7 and the Y412MC61 attP site has a nucleotide sequence that is at least 98% identical to SEQ ID NO: 8. In some embodiments, the Y412MC61 attB site has a nucleotide sequence that is at least 99% identical to SEQ ID NO: 7 and the Y412MC61 attP site has a nucleotide sequence that is at least 99% identical to SEQ ID NO: 8. In some embodiments, the Y412MC61 attB site has a nucleotide sequence as set forth in SEQ ID NO: 7 and the Y412MC61 attP site has a nucleotide sequence as set forth in SEQ ID NO: 8.

SEQ ID NO: 7
agacgagaaacgttccgtccgtctgggtcagttgggcaaagttgatgac
cgggtcgtccgtt
SEQ ID NO: 8
gtgttataaacctgtgtgagagttaagtttacatgcctaaccttaactt
ttacgcaggttcagctt

In some embodiments, the pair of attBlattP recombination sites is specific to a BXB1 recombinase. In some embodiments, the attB and attP recombination sites each have a nucleotide sequence specific to a BXB1 recombinase. In some embodiments, the BXB1 attB site has a nucleotide sequence that is at least 90% identical to SEQ ID NO: 9 and the BXB1 attP site has a nucleotide sequence that is at least 90% identical to SEQ ID NO: 10. In some embodiments, the BXB1 attB site has a nucleotide sequence that is at least 91% identical to SEQ ID NO: 9 and the BXB1 attP site has a nucleotide sequence that is at least 91% identical to SEQ ID NO: 10. In some embodiments, the BXB1 attB site has a nucleotide sequence that is at least 92% identical to SEQ ID NO: 9 and the BXB1 attP site has a nucleotide sequence that is at least 92% identical to SEQ ID NO: 10. In some embodiments, the BXB1 attB site has a nucleotide sequence that is at least 93% identical to SEQ ID NO: 9 and the BXB1 attP site has a nucleotide sequence that is at least 93% identical to SEQ ID NO: 10. In some embodiments, the BXB1 attB site has a nucleotide sequence that is at least 94% identical to SEQ ID NO: 9 and the BXB1 attP site has a nucleotide sequence that is at least 94% identical to SEQ ID NO: 10. In some embodiments, the BXB1 attB site has a nucleotide sequence that is at least 95% identical to SEQ ID NO: 9 and the BXB1 attP site has a nucleotide sequence that is at least 95% identical to SEQ ID NO: 10. In some embodiments, the BXB1 attB site has a nucleotide sequence that is at least 96% identical to SEQ ID NO: 9 and the BXB1 attP site has a nucleotide sequence that is at least 96% identical to SEQ ID NO: 10. In some embodiments, the BXB1 attB site has a nucleotide sequence that is at least 97% identical to SEQ ID NO: 9 and the BXB1 attP site has a nucleotide sequence that is at least 97% identical to SEQ ID NO: 10. In some embodiments, the BXB1 attB site has a nucleotide sequence that is at least 98% identical to SEQ ID NO: 9 and the BXB1 attP site has a nucleotide sequence that is at least 98% identical to SEQ ID NO: 10. In some embodiments, the BXB1 attB site has a nucleotide sequence that is at least 99% identical to SEQ ID NO: 9 and the BXB1 attP site has a nucleotide sequence that is at least 99% identical to SEQ ID NO: 10. In some embodiments, the BXB1 attB site has a nucleotide sequence as set forth in SEQ ID NO: 9 and the BXB1 attP site has a nucleotide sequence as set forth in SEQ ID NO: 10.

SEQ ID NO: 9
tcggccggcttgtcgacgacggcggtctccgtcgtcaggatcatccggg
c
SEQ ID NO: 10
gtcgtggtttgtctggtcaaccaccgcggtctcagtggtgtacggtaca
aaccccgac

In some embodiments, the pair of attBlattP recombination sites is specific to a TG1 recombinase. In some embodiments, the attB and attP recombination sites each have a nucleotide sequence specific to a TG1 recombinase. In some embodiments, the TG1 attB site has a nucleotide sequence that is at least 90% identical to SEQ ID NO: 11 and the TG1 attP site has a nucleotide sequence that is at least 90% identical to SEQ ID NO: 12. In some embodiments, the TG1 attB site has a nucleotide sequence that is at least 91% identical to SEQ ID NO: 11 and the TG1 attP site has a nucleotide sequence that is at least 91% identical to SEQ ID NO: 12. In some embodiments, the TG1 attB site has a nucleotide sequence that is at least 92% identical to SEQ ID NO: 11 and the TG1 attP site has a nucleotide sequence that is at least 92% identical to SEQ ID NO: 12. In some embodiments, the TG1 attB site has a nucleotide sequence that is at least 93% identical to SEQ ID NO: 11 and the TG1 attP site has a nucleotide sequence that is at least 93% identical to SEQ ID NO: 12. In some embodiments, the TG1 attB site has a nucleotide sequence that is at least 94% identical to SEQ ID NO: 11 and the TG1 attP site has a nucleotide sequence that is at least 94% identical to SEQ ID NO: 12. In some embodiments, the TG1 attB site has a nucleotide sequence that is at least 95% identical to SEQ ID NO: 11 and the TG1 attP site has a nucleotide sequence that is at least 95% identical to SEQ ID NO: 12. In some embodiments, the TG1 attB site has a nucleotide sequence that is at least 96% identical to SEQ ID NO: 11 and the TG1 attP site has a nucleotide sequence that is at least 96% identical to SEQ ID NO: 12. In some embodiments, the TG1 attB site has a nucleotide sequence that is at least 97% identical to SEQ ID NO: 11 and the TG1 attP site has a nucleotide sequence that is at least 97% identical to SEQ ID NO: 12. In some embodiments, the TG1 attB site has a nucleotide sequence that is at least 98% identical to SEQ ID NO: 11 and the TG1 attP site has a nucleotide sequence that is at least 98% identical to SEQ ID NO: 12. In some embodiments, the TG1 attB site has a nucleotide sequence that is at least 99% identical to SEQ ID NO: 11 and the TG1 attP site has a nucleotide sequence that is at least 99% identical to SEQ ID NO: 12. In some embodiments, the TG1 attB site has a nucleotide sequence as set forth in SEQ ID NO: 11 and the TG1 attP site has a nucleotide sequence as set forth in SEQ ID NO: 12.

SEQ ID NO: 11
gatcagctccgcgggcaagaccttctccttcacggggtggaaggtc
SEQ ID NO: 12
tcaaccccgttccagcccaacagtgttagtctttgctcttacccagttg
ggcgggatagcctgcccg

In some embodiments, there are multiple att pairs in the bacterial genome. In such embodiments, the multiple pairs are specific to different thermophilic recombinases. Therefore, the sequences of each att pair are different from the other att pairs. For example, a bacterial genome may have two att recombination sites, one is specific to a thermophilic recombinase while the other att recombination site is specific to a different thermophilic recombinase. As such, the two att recombination sites of the bacterial genome comprise different nucleic acid sequences from each other. In some embodiments, one pair of the multiple att pairs is specific to Y412MC61 recombinase, one pair of the multiple att pairs is specific to BXB1 recombinase, and one pair of the multiple att pairs is specific to TG1 recombinase. In some embodiments, one pair of the multiple att pairs is specific to Y412MC61 recombinase and one pair of the multiple att pairs is specific to BXB1 recombinase. In some embodiments, one pair of the multiple att pairs is specific to Y412MC61 recombinase and one pair of the multiple att pairs is specific to TG1 recombinase. In some embodiments, one pair of the multiple att pairs is specific to BXB1 recombinase and one pair of the multiple att pairs is specific to TG1 recombinase.

As used herein, a “scar site” is a site where the recombination did occur, i.e. the recombination site after the recombination has taken place. The scar site occurs in the genome of the bacterial cell. Unlike the site where the recombination did occur via Cre and Flp recombinases, the scar site of the site-specific recombinases of this disclosure cannot act as a substrate for further recombination, making the recombination event irreversible and stable. In some embodiments, the recombination that occurs between the attB and attP sites results in the formation of new attL and attR sites. These attL and an attR scar sites are considered genetic scars.

In some embodiments, the pair of attL/attR scar sites is created by a Y412MC61 recombinase. In some embodiments, the Y412MC61 attL site has a nucleotide sequence that is at least 90% identical to SEQ ID NO: 13 and the Y412MC61 attR site has a nucleotide sequence that is at least 90% identical to SEQ ID NO: 14. In some embodiments, the Y412MC61 attL site has a nucleotide sequence that is at least 91% identical to SEQ ID NO: 13 and the Y412MC61 attR site has a nucleotide sequence that is at least 91% identical to SEQ ID NO: 14. In some embodiments, the Y412MC61 attL site has a nucleotide sequence that is at least 92% identical to SEQ ID NO: 13 and the Y412MC61 attR site has a nucleotide sequence that is at least 92% identical to SEQ ID NO: 14. In some embodiments, the Y412MC61 attL site has a nucleotide sequence that is at least 93% identical to SEQ ID NO: 13 and the Y412MC61 attR site has a nucleotide sequence that is at least 93% identical to SEQ ID NO: 14. In some embodiments, the Y412MC61 attL site has a nucleotide sequence that is at least 94% identical to SEQ ID NO: 13 and the Y412MC61 attR site has a nucleotide sequence that is at least 94% identical to SEQ ID NO: 14. In some embodiments, the Y412MC61 attL site has a nucleotide sequence that is at least 95% identical to SEQ ID NO: 13 and the Y412MC61 attR site has a nucleotide sequence that is at least 95% identical to SEQ ID NO: 14. In some embodiments, the Y412MC61 attL site has a nucleotide sequence that is at least 96% identical to SEQ ID NO: 13 and the Y412MC61 attR site has a nucleotide sequence that is at least 96% identical to SEQ ID NO: 14. In some embodiments, the Y412MC61 attL site has a nucleotide sequence that is at least 97% identical to SEQ ID NO: 13 and the Y412MC61 attR site has a nucleotide sequence that is at least 97% identical to SEQ ID NO: 14. In some embodiments, the Y412MC61 attL site has a nucleotide sequence that is at least 98% identical to SEQ ID NO: 13 and the Y412MC61 attR site has a nucleotide sequence that is at least 98% identical to SEQ ID NO: 14. In some embodiments, the Y412MC61 attL site has a nucleotide sequence that is at least 99% identical to SEQ ID NO: 13 and the Y412MC61 attR site has a nucleotide sequence that is at least 99% identical to SEQ ID NO: 14. In some embodiments, the Y412MC61 attL site has a nucleotide sequence of SEQ ID NO: 13 and the Y412MC61 attR site has a nucleotide sequence of SEQ ID NO: 14.

SEQ ID NO: 13
agacgagaaacgttccgtccgtctgggtcagttgcctaaccttaacttt
tacgcaggttcagctt
SEQ ID NO: 14
gtgttataaacctgtgtgagagttaagtttacatgggcaaagttgatga
ccgggtcgtccgtt

In some embodiments, the pair of attL/attR scar sites is created by a BXB1 recombinase. In some embodiments, the BXB1 attL site has a nucleotide sequence that is at least 50% identical to SEQ ID NO: 15 and the BXB1 attR site has a nucleotide sequence that is at least 50% identical to SEQ ID NO: 16. In some embodiments, the BXB1 attL site has a nucleotide sequence that is at least 60% identical to SEQ ID NO: 15 and the BXB1 attR site has a nucleotide sequence that is at least 60% identical to SEQ ID NO: 16. In some embodiments, the BXB1 attL site has a nucleotide sequence that is at least 70% identical to SEQ ID NO: 15 and the BXB1 attR site has a nucleotide sequence that is at least 70% identical to SEQ ID NO: 16. In some embodiments, the BXB1 attL site has a nucleotide sequence that is at least 80% identical to SEQ ID NO: 15 and the BXB1 attR site has a nucleotide sequence that is at least 80% identical to SEQ ID NO: 16. In some embodiments, the BXB1 attL site has a nucleotide sequence that is at least 90% identical to SEQ ID NO: 15 and the BXB1 attR site has a nucleotide sequence that is at least 90% identical to SEQ ID NO: 16. In some embodiments, the BXB1 attL site has a nucleotide sequence that is at least 91% identical to SEQ ID NO: 15 and the BXB1 attR site has a nucleotide sequence that is at least 91% identical to SEQ ID NO: 16. In some embodiments, the BXB1 attL site has a nucleotide sequence that is at least 92% identical to SEQ ID NO: 15 and the BXB1 attR site has a nucleotide sequence that is at least 92% identical to SEQ ID NO: 16. In some embodiments, the BXB1 attL site has a nucleotide sequence that is at least 93% identical to SEQ ID NO: 15 and the BXB1 attR site has a nucleotide sequence that is at least 93% identical to SEQ ID NO: 16. In some embodiments, the BXB1 attL site has a nucleotide sequence that is at least 94% identical to SEQ ID NO: 15 and the BXB1 attR site has a nucleotide sequence that is at least 94% identical to SEQ ID NO: 16. In some embodiments, the BXB1 attL site has a nucleotide sequence that is at least 95% identical to SEQ ID NO: 15 and the BXB1 attR site has a nucleotide sequence that is at least 95% identical to SEQ ID NO: 16. In some embodiments, the BXB1 attL site has a nucleotide sequence that is at least 96% identical to SEQ ID NO: 15 and the BXB1 attR site has a nucleotide sequence that is at least 96% identical to SEQ ID NO: 16. In some embodiments, the BXB1 attL site has a nucleotide sequence that is at least 97% identical to SEQ ID NO: 15 and the BXB1 attR site has a nucleotide sequence that is at least 97% identical to SEQ ID NO: 16. In some embodiments, the BXB1 attL site has a nucleotide sequence that is at least 98% identical to SEQ ID NO: 15 and the BXB1 attR site has a nucleotide sequence that is at least 98% identical to SEQ ID NO: 16. In some embodiments, the BXB1 attL site has a nucleotide sequence that is at least 99% identical to SEQ ID NO: 15 and the BXB1 attR site has a nucleotide sequence that is at least 99% identical to SEQ ID NO: 16. In some embodiments, the BXB1 attL site has a nucleotide sequence of SEQ ID NO: 15 and the BXB1 attR site has a nucleotide sequence of SEQ ID NO: 16.

SEQ ID NO: 15
tcggccggcttgtcgacgacggcggtctcagtggtgtacggtacaaacc
ccgac
SEQ ID NO: 16
gtcgtggtttgtctggtcaaccaccgcggtctccgtcgtcaggatcatc
cgggc

In some embodiments, the pair of attL/attR scar sites is created by a TG1 recombinase. In some embodiments, the TG1 attL site has a nucleotide sequence that is at least 50% identical to SEQ ID NO: 17 and the TG1 attR site has a nucleotide sequence that is at least 50% identical to SEQ ID NO: 18. In some embodiments, the TG1 attL site has a nucleotide sequence that is at least 60% identical to SEQ ID NO: 17 and the TG1 attR site has a nucleotide sequence that is at least 60% identical to SEQ ID NO: 18. In some embodiments, the TG1 attL site has a nucleotide sequence that is at least 70% identical to SEQ ID NO: 17 and the TG1 attR site has a nucleotide sequence that is at least 70% identical to SEQ ID NO: 18. In some embodiments, the TG1 attL site has a nucleotide sequence that is at least 80% identical to SEQ ID NO: 17 and the TG1 attR site has a nucleotide sequence that is at least 80% identical to SEQ ID NO: 18. In some embodiments, the TG1 attL site has a nucleotide sequence that is at least 90% identical to SEQ ID NO: 17 and the TG1 attR site has a nucleotide sequence that is at least 90% identical to SEQ ID NO: 18. In some embodiments, the TG1 attL site has a nucleotide sequence that is at least 91% identical to SEQ ID NO: 17 and the TG1 attR site has a nucleotide sequence that is at least 91% identical to SEQ ID NO: 18. In some embodiments, the TG1 attL site has a nucleotide sequence that is at least 92% identical to SEQ ID NO: 17 and the TG1 attR site has a nucleotide sequence that is at least 92% identical to SEQ ID NO: 18. In some embodiments, the TG1 attL site has a nucleotide sequence that is at least 93% identical to SEQ ID NO: 17 and the TG1 attR site has a nucleotide sequence that is at least 93% identical to SEQ ID NO: 18. In some embodiments, the TG1 attL site has a nucleotide sequence that is at least 94% identical to SEQ ID NO: 17 and the TG1 attR site has a nucleotide sequence that is at least 94% identical to SEQ ID NO: 18. In some embodiments, the TG1 attL site has a nucleotide sequence that is at least 95% identical to SEQ ID NO: 17 and the TG1 attR site has a nucleotide sequence that is at least 95% identical to SEQ ID NO: 18. In some embodiments, the TG1 attL site has a nucleotide sequence that is at least 96% identical to SEQ ID NO: 17 and the TG1 attR site has a nucleotide sequence that is at least 96% identical to SEQ ID NO: 18. In some embodiments, the TG1 attL site has a nucleotide sequence that is at least 97% identical to SEQ ID NO: 17 and the TG1 attR site has a nucleotide sequence that is at least 97% identical to SEQ ID NO: 18. In some embodiments, the TG1 attL site has a nucleotide sequence that is at least 98% identical to SEQ ID NO: 17 and the TG1 attR site has a nucleotide sequence that is at least 98% identical to SEQ ID NO: 18. In some embodiments, the TG1 attL site has a nucleotide sequence that is at least 99% identical to SEQ ID NO: 17 and the TG1 attR site has a nucleotide sequence that is at least 99% identical to SEQ ID NO: 18. In some embodiments, the TG1 attL site has a nucleotide sequence of SEQ ID NO: 17 and the TG1 attR site has a nucleotide sequence of SEQ ID NO: 18.

SEQ ID NO: 17
gatcagctccgcgggcaagacctttgctcttacccagttgggcgggata
gcctgcccg
SEQ ID NO: 18
tcaaccccgttccagcccaacagtgttagtcttctccttcacggggtgg
aaggtc

Thermophiles are heat-loving organisms that exhibit optimal growth at a temperature at or above 50° C. As such, thermophilic bacteria are bacteria which grow and thrive at temperatures of 50° C. or more. There are several known thermophilic bacteria known in the art. Thermophiles are inhabitants of various ecological niches like deep sea hydrothermal vents, terrestrial hot springs, and other extreme geographical/geological sites including volcanic sites, tectonically active faults as well as decaying matters such as the compost and deep organic landfills. Non-limiting examples of thermophilic bacteria include bacteria from the genus of: Bacillus, Geobacillus, Paenebacillus, Clostridium, Anaerocellum, Caldicellulosiruptor, Thermus, Pyrococcus, Thermococcus, Thermoanaerobacter, Thermoplasma, Thermosipho, Thermoanaerobacterium, Herbinix, Acetivibrio, Acidothermus, Hydrogenobaculum, Rhodoplanes, Ornithinibacillus, Thermaerobacter, Fervidobacterium, and Persephonella, among others.

### Genetically Engineered Thermophile Bacterial Cell For Receiving a DNA Insertion

One aspect of the present disclosure is directed to a genetically engineered thermophile bacterial cell comprising at least one att site in its chromosome wherein the att site is one member of a pair of attB and attP recombination sites. Such bacterial cell is useful as a recipient cell for insertion of a DNA. In some embodiments, the att site in the genetically engineered thermophile bacterial cell is an attB recombination site. In some embodiments, the att site in the genetically engineered thermophile bacterial cell is an attP recombination site. In some embodiments, the cell expresses a thermophilic site-specific recombinase that recognizes the pair of attB and attP recombination sites. In some embodiments, the at least one att site comprises multiple att sites, each being a member of a pair of attB and attP recombination sites recognized by different site-specific recombinases.

In some embodiments, the genetically engineered thermophile bacterial cell is prepared through the insertion of an att site into the chromosome. Methods of preparation are known in the art, any of which can be used. In some embodiments, the att site was inserted through homologous recombination, wherein a plasmid is transformed into the bacterial chromosome, the transformation is confirmed through PCR, and isolates with confirmed genomic integration were selected.

### System for Insertion of DNA

One aspect of the current disclosure is directed to a system for stable insertion of a heterologous DNA, the system comprising:
the thermophile bacterial cell comprising at least one att site in its chromosome wherein the att site is one member of a pair of attB and attP recombination sites; and
a cargo plasmid comprising the heterologous DNA and a corresponding att site, wherein the corresponding att site is the other member of the pair of attB and attP recombination sites;
wherein the cargo plasmid is capable of insertion into the chromosome of the thermophile at the at least one att site via recombination with the corresponding att site on the cargo plasmid; and
wherein the recombination is mediated by a thermophilic site-specific recombinase that recognizes the pair of attB and attP recombination sites, and results in the formation of an attL site and an attR site, wherein the attL and attR sites are not substrates for further recombination.

As used herein, a “system” refers to a combination of multiple components or products which interact in a way to produce a desired result. In some embodiments, the components could be provided in the form of a kit. In some embodiments, the disclosure provides kits containing any one or more of the elements disclosed in the above methods and compositions. In some embodiments, the kit comprises a vector system and instructions for using the kit.

In some embodiment the thermophile bacterial cell includes a native att site in its chromosome. For example, some Geobacillus strains have a native att site in their genome. In some embodiments, the thermophile bacterial cell is a genetically engineered bacterial cell. In some embodiments, the thermophile bacterial cell is genetically engineered to include one or more att sites in its chromosome. In some embodiments, the thermophile bacterial cell comprises both native att and genetically engineered att sites.

In some embodiments, the thermophile bacterial cell expresses the thermophilic site-specific recombinase. As used herein, “expressing” or “expresses” refer to the production of a functional end-product e.g., an mRNA or a protein (precursor or mature) by the transformed cell. In some embodiments, the disclosed system further comprises a nucleic acid encoding the thermophilic site-specific recombinase. In some embodiments, the nucleic acid encoding the thermophilic site-specific recombinase is provided on a helper plasmid.

In some embodiments, the cargo plasmid further comprises a selectable marker gene. A selectable marker gene is a gene expressing a protein that allows for selection of a cell which has undergone successful transformation. In some embodiments, the selectable marker gene is flanked by another pair of attB and attP recombination sites recognized by another thermophilic site-specific recombinase. In some embodiments, the thermophilic site-specific recombinase and the another thermophilic site-specific recombinase are a serine recombinase or a tyrosine recombinase. For example, the att pair used for integration of the cargo plasmid into the bacterial chromosome is an attB/attP pair specific to a Y412MC61 recombinase, while another pair of att recombination sites is a pair of attBlattP recombination sites which flank the selectable marker gene that is located in the cargo DNA which gets inserted into the bacterial chromosome. The additional pair of att recombination sites is a pair of attBlattP recombination sites that are specific to the BXB1 recombinase. As such, the additional pair of att recombination sites flanking the selectable marker gene allows for the selectable marker gene to be excised from the bacterial chromosome through the use of BXB1 recombinase once the selectable marker is no longer needed.

Thermophilic site-specific recombinases are defined supra. In some embodiments, the thermophilic site-specific recombinase is a recombinase that is able to serve its function at thermophilic temperatures. In some embodiments, the thermophilic site-specific recombinase is a tyrosine or serine recombinase. In some embodiments, the serine recombinase is selected from Y412MC61, BXB1, qBT1, qFC1, qRV1, TG1, R4, BL3, qA118, qMR11, and q370 recombinases and homologs thereof. In some embodiments, the serine recombinase is selected from Y412MC61, BXB1, and TG1, and homologs thereof.

In some embodiments, the nucleic acid encoding the serine recombinase is a nucleic acid encoding the Y412MC61 recombinase. In some embodiments, the nucleic acid encoding the serine recombinase is a nucleic acid encoding a Y412MC61 recombinase homolog and comprising a nucleotide sequence having at least 90% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 91% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 92% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 93% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 94% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 95% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 96% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 97% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 98% sequence identity to the nucleotide sequence shown in SEQ ID NO: 1. In some embodiments, the nucleic acid encoding the serine recombinase is a nucleic acid encoding a Y412MC61 recombinase homolog and comprising a nucleotide sequence having at least 99% sequence identity to the nucleotide sequence shown in SEQ ID NO: 1. In some embodiments, the nucleic acid encoding the serine recombinase is a nucleic acid encoding the Y412MC61 recombinase comprising a nucleotide sequence of SEQ ID NO: 1.

SEQ ID NO: 1
atgaaagtggccatttatgttcgtgttagcaccgatgaacaggccaaag
aaggttttagcattccggcacagcgtgaacgtctgcgtgcattttgtgc
aagccagggttgggaaattgtgcaagaatatattgaagaaggttggagc
gcaaaagatctggatcgtccgcagatgcagcgtctgctgaaagatatca
aaaaaggcaacattgatattgtgctggtgtatcgtctggatcgcctgac
ccgtagcgttctggatctgtatctgctgctgcagacctttgaaaaatac
aatgtggcatttcgtagcgccaccgaagtttatgataccagcaccgcaa
tgggtcgtctgtttattaccctggttgcagcactggcacagtgggaacg
tgaaaatctggcagaacgtgttaaatttggtatcgagcagatgatcgat
gaaggtaaaaaaccgggtggtcatagcccgtatggttacaaatttgata
aagacttcaattgcaccattattgaggaagaagcagacgttgttcgtat
gatctatcgcatgtattgtgatggttatggctatcgtagcattgcagat
cgtctgaatgaactgatggttaaaccgcgtattgccaaagaatggaatc
ataatagcgtgcgtgatatcctgaccaacgatatctatattggcaccta
tcgttggggtgataaagttgttccgaataatcatccgcctattattagc
gaaaccctgttcaaaaaagcccagaaagaaaaagaaaaacgtggcgttg
atcgtaaacgcgttggtaaatttctgtttaccggtctgctgcagtgtgg
taattgtggtggccataaaatgcagggccattttgataaacgtgagcag
aaaacctattaccgttgtaccaaatgtcaccgcattaccaacgaaaaaa
acattctggaaccgctgctggatgaaattcagctgctgattaccagcaa
agaatactttatgagcaaattcagcgaccgctatgatcagcaagaggtt
gttgatgttagcgcactgacaaaagaactggaaaaaatcaaacgccaga
aagagaaatggtacgatctgtatatggatgatcgtaacccgattccgaa
agaagaactgtttgccaaaattaacgaactgaacaaaaaagaagaagaa
atctatagcaagctgagcgaagtggaagaagataaagaaccggttgaag
agaaatataaccgcctgagcaaaatgatcgattttaaacagcagtttga
gcaggccaacgactttaccaaaaaagagctgctgttcagcatcttcgaa
aagattgtgatttatcgcgagaaaggcaagctgaaaaaaatcaccctgg
attacaccctgaaataa

In some embodiments, the Y412MC61 recombinase comprises an amino acid sequence of SEQ ID NO: 2. In some embodiments, the aY412MC61 recombinase homolog comprises an amino acid sequence having at least 90% sequence identity to the amino acid sequence shown in SEQ ID NO: 2. In some embodiments, the a Y412MC61 recombinase homolog comprises an amino acid sequence having at least 91% sequence identity to the amino acid sequence shown in SEQ ID NO: 2. In some embodiments, the aY412MC61 recombinase homolog comprises an amino acid sequence having at least 92% sequence identity to the amino acid sequence shown in SEQ ID NO: 2. In some embodiments, the aY412MC61 recombinase homolog comprises an amino acid sequence having at least 93% sequence identity to the amino acid sequence shown in SEQ ID NO: 2. In some embodiments, the aY412MC61 recombinase homolog comprises an amino acid sequence having at least 94% sequence identity to the amino acid sequence shown in SEQ ID NO: 2. In some embodiments, the aY412MC61 recombinase homolog comprises an amino acid sequence having at least 95% sequence identity to the amino acid sequence shown in SEQ ID NO: 2. In some embodiments, the aY412MC61 recombinase homolog comprises an amino acid sequence having at least 96% sequence identity to the amino acid sequence shown in SEQ ID NO: 2. In some embodiments, the aY412MC61 recombinase homolog comprises an amino acid sequence having at least 97% sequence identity to the amino acid sequence shown in SEQ ID NO: 2. In some embodiments, the aY412MC61 recombinase homolog comprises an amino acid sequence having at least 98% sequence identity to the amino acid sequence shown in SEQ ID NO: 2. In some embodiments, the aY412MC61 recombinase homolog comprises an amino acid sequence having at least 99% sequence identity to the amino acid sequence shown in SEQ ID NO: 2.

SEQ ID NO: 2
MKVAIYVRVSTDEQAKEGFSIPAQRERLRAFCASQGWEIVQEYIEEGWS
AKDLDRPQMQRLLKDIKKGNIDIVLVYRLDRLTRSVLDLYLLLQTFEKY
NVAFRSATEVYDTSTAMGRLFITLVAALAQWERENLAERVKFGIEQMID
EGKKPGGHSPYGYKFDKDFNCTIIEEEADVVRMIYRMYCDGYGYRSIAD
RLNELMVKPRIAKEWNHNSVRDILTNDIYIGTYRWGDKVVPNNHPPIIS
ETLFKKAQKEKEKRGVDRKRVGKFLFTGLLQCGNCGGHKMQGHFDKREQ
KTYYRCTKCHRITNEKNILEPLLDEIQLLITSKEYFMSKFSDRYDQQEV
VDVSALTKELEKIKRQKEKWYDLYMDDRNPIPKEELFAKINELNKKEEE
IYSKLSEVEEDKEPVEEKYNRLSKMIDFKQQFEQANDFTKKELLFSIFE
KIVIYREKGKLKKITLDYTLK

In some embodiments, the nucleic acid encoding the serine recombinase is a nucleic acid encoding the BXB1 recombinase or homologues thereof. In some embodiments, the nucleic acid encoding the serine recombinase is a nucleic acid encoding a BXB1 recombinase homolog and comprising a nucleotide sequence having at least 90% sequence identity to SEQ ID NO: 3. In some embodiments, the nucleic acid encoding the serine recombinase is a nucleic acid encoding a BXB1 recombinase homolog and comprising a nucleotide sequence having at least 91% sequence identity to SEQ ID NO: 3. In some embodiments, the nucleic acid encoding the serine recombinase is a nucleic acid encoding a BXB1 recombinase homolog and comprising a nucleotide sequence having at least 92% sequence identity to SEQ ID NO: 3. In some embodiments, the nucleic acid encoding the serine recombinase is a nucleic acid encoding a BXB1 recombinase homolog and comprising a nucleotide sequence having at least 93% sequence identity to SEQ ID NO: 3. In some embodiments, the nucleic acid encoding the serine recombinase is a nucleic acid encoding a BXB1 recombinase homolog and comprising a nucleotide sequence having at least 94% sequence identity to SEQ ID NO: 3. In some embodiments, the nucleic acid encoding the serine recombinase is a nucleic acid encoding a BXB1 recombinase homolog and comprising a nucleotide sequence having at least 95% sequence identity to SEQ ID NO: 3. In some embodiments, the nucleic acid encoding the serine recombinase is a nucleic acid encoding a BXB1 recombinase homolog and comprising a nucleotide sequence having at least 96% sequence identity to SEQ ID NO: 3. In some embodiments, the nucleic acid encoding the serine recombinase is a nucleic acid encoding a BXB1 recombinase homolog and comprising a nucleotide sequence having at least 97% sequence identity to SEQ ID NO: 3. In some embodiments, the nucleic acid encoding the serine recombinase is a nucleic acid encoding a BXB1 recombinase homolog and comprising a nucleotide sequence having at least 98% sequence identity to SEQ ID NO: 3. In some embodiments, the nucleic acid encoding the serine recombinase is a nucleic acid encoding a BXB1 recombinase homolog and comprising a nucleotide sequence having at least 99% sequence identity to SEQ ID NO: 3. In some embodiments, the nucleic acid encoding the serine recombinase is a nucleic acid encoding the BXB1 recombinase comprising a nucleotide sequence of SEQ ID NO: 3.

SEQ ID NO: 3
atgagagctttggttgtgatcagacttagtagagtaacagatgcgacta
cttctcctgaaaggcagttggaaagttgtcaacaactctgcgctcaaag
aggatgggatgtcgttggagttgcagaggatttagatgtatccggcgct
gttgaccctttcgatagaaaaagaagacctaatttagcaagatggctag
catttgaagagcagccttttgatgttatagttgcctatagagttgacag
attaacaagatctataagacaccttcaacaattagtccattgggcagag
gatcataaaaaacttgtggtttcggctacagaagcacattttgatacaa
caaccccatttgctgcagtagttattgccttaatgggaacagttgctca
aatggaacttgaagctattaaggaaagaaatagaagtgctgcccacttt
aatataagagcgggaaagtacagaggatcactccctccttggggatatc
tacctactagggtagatggagaatggaggctcgtgccagatccagttca
aagagaaagaatattagaagtatatcatagagtagtagacaatcatgag
cctcttcatcttgtagctcatgatctaaaccgacgaggcgtgttatcac
ctaaggattattttgcccagctacagggcagggagcctcaaggaagaga
gtggagtgctacagctttaaagaggtctatgataagcgaagctatgctt
gggtatgctaccttaaatggaaaaaccgtaagagatgatgatggtgctc
cattagttagagctgaaccaattcttactagagaacaattggaagcact
tagggcagagcttgtaaaaactagccgagcaaaacctgctgtttctact
ccatctctgctgcttagagttttattttgtgctgtttgcggtgagccag
catataaatttgcaggtggaggaagaaagcacccaagatatagatgtag
aagcatgggttttccaaaacattgtggaaatggtacagtagcaatggca
gagtgggatgcattctgtgaagaacaagttttggatttattaggagatg
ctgaacgtcttgaaaaagtatgggttgcgggtagtgattctgcagtaga
attagctgaagtaaacgctgaattagtagatttaactagtttaataggt
tcacccgcgtaccgggcaggatcaccacagagagaagctctcgatgcaa
ggattgcagcattagcagctagacaagaagagcttgaaggtttagaggc
aagaccgtcaggttgggaatggagagaaacggggcaaagatttggtgat
tggtggagagaacaagatacggcagctaaaaatacttggttaagatcaa
tgaatgttagattaacttttgatgtacgcggcggattgacaagaacaat
agattttggagaccttcaggaatatgaacaacatcttcgtcttggttca
gtagttgaaagactacatacaggaatgagttaa

In some embodiments, the BXB1 recombinase comprises an amino acid sequence of SEQ ID NO: 4. In some embodiments, the BXB1 recombinase homolog comprises an amino acid sequence having at least 90% sequence identity to the amino acid sequence shown in SEQ ID NO: 4. In some embodiments, the BXB1 recombinase homolog comprises an amino acid sequence having at least 91% sequence identity to the amino acid sequence shown in SEQ ID NO: 4. In some embodiments, the BXB1 recombinase homolog comprises an amino acid sequence having at least 92% sequence identity to the amino acid sequence shown in SEQ ID NO: 4. In some embodiments, the BXB1 recombinase homolog comprises an amino acid sequence having at least 93% sequence identity to the amino acid sequence shown in SEQ ID NO: 4. In some embodiments, the BXB1 recombinase homolog comprises an amino acid sequence having at least 94% sequence identity to the amino acid sequence shown in SEQ ID NO: 4. In some embodiments, the BXB1 recombinase homolog comprises an amino acid sequence having at least 95% sequence identity to the amino acid sequence shown in SEQ ID NO: 4. In some embodiments, the BXB1 recombinase homolog comprises an amino acid sequence having at least 96% sequence identity to the amino acid sequence shown in SEQ ID NO: 4. In some embodiments, the BXB1 recombinase homolog comprises an amino acid sequence having at least 97% sequence identity to the amino acid sequence shown in SEQ ID NO: 4. In some embodiments, the BXB1 recombinase homolog comprises an amino acid sequence having at least 98% sequence identity to the amino acid sequence shown in SEQ ID NO: 4. In some embodiments, the BXB1 recombinase homolog comprises an amino acid sequence having at least 99% sequence identity to the amino acid sequence shown in SEQ ID NO: 4.

SEQ ID NO: 4
MRALVVIRLSRVTDATTSPERQLESCQQLCAQRGWDVVGVAEDLDVSGA
VDPFDRKRRPNLARWLAFEEQPFDVIVAYRVDRLTRSIRHLQQLVHWAE
DHKKLVVSATEAHFDTTTPFAAVVIALMGTVAQMELEAIKERNRSAAHF
NIRAGKYRGSLPPWGYLPTRVDGEWRLVPDPVQRERILEVYHRVVDNHE
PLHLVAHDLNRRGVLSPKDYFAQLQGREPQGREWSATALKRSMISEAML
GYATLNGKTVRDDDGAPLVRAEPILTREQLEALRAELVKTSRAKPAVST
PSLLLRVLFCAVCGEPAYKFAGGGRKHPRYRCRSMGFPKHCGNGTVAMA
EWDAFCEEQVLDLLGDAERLEKVWVAGSDSAVELAEVNAELVDLTSLIG
SPAYRAGSPQREALDARIAALAARQEELEGLEARPSGWEWRETGQRFGD
WWREQDTAAKNTWLRSMNVRLTFDVRGGLTRTIDFGDLQEYEQHLRLGS
VVERLHTGMS

In some embodiments, the nucleic acid encoding the serine recombinase is a nucleic acid encoding the TG1 recombinase or a homologue thereof. In some embodiments, the nucleic acid encoding the serine recombinase is a nucleic acid encoding a TG1 recombinase homolog and comprising a nucleotide sequence having at least 90% sequence identity to SEQ ID NO: 5. In some embodiments, the nucleic acid encoding the serine recombinase is a nucleic acid encoding a TG1 recombinase homolog and comprising a nucleotide sequence having at least 91% sequence identity to SEQ ID NO: 5. In some embodiments, the nucleic acid encoding the serine recombinase is a nucleic acid encoding a TG1 recombinase homolog and comprising a nucleotide sequence having at least 92% sequence identity to SEQ ID NO: 5. In some embodiments, the nucleic acid encoding the serine recombinase is a nucleic acid encoding a TG1 recombinase homolog and comprising a nucleotide sequence having at least 93% sequence identity to SEQ ID NO: 5. In some embodiments, the nucleic acid encoding the serine recombinase is a nucleic acid encoding a TG1 recombinase homolog and comprising a nucleotide sequence having at least 94% sequence identity to SEQ ID NO: 5. In some embodiments, the nucleic acid encoding the serine recombinase is a nucleic acid encoding a TG1 recombinase homolog and comprising a nucleotide sequence having at least 95% sequence identity to SEQ ID NO: 5. In some embodiments, the nucleic acid encoding the serine recombinase is a nucleic acid encoding a TG1 recombinase homolog and comprising a nucleotide sequence having at least 96% sequence identity to SEQ ID NO: 5. In some embodiments, the nucleic acid encoding the serine recombinase is a nucleic acid encoding a TG1 recombinase homolog and comprising a nucleotide sequence having at least 97% sequence identity to SEQ ID NO: 5. In some embodiments, the nucleic acid encoding the serine recombinase is a nucleic acid encoding a TG1 recombinase homolog and comprising a nucleotide sequence having at least 98% sequence identity to SEQ ID NO: 5. In some embodiments, the nucleic acid encoding the serine recombinase is a nucleic acid encoding a TG1 recombinase homolog and comprising a nucleotide sequence having at least 99% sequence identity to SEQ ID NO: 5. In some embodiments, the nucleic acid encoding the serine recombinase is a nucleic acid encoding the TG1 recombinase comprising a nucleotide sequence of SEQ ID NO: 5.

SEQ ID NO: 5
atggttattttagctggtggatatgatagacagtcggcagaacgtgaaa
attcaagtacagcttctccagcaacacaaagagctgcaaatagaggtaa
ggcagaagctctagcaaaagaatatgcaagggatggtgtagaagttaaa
tggcttggacattttagtgaggcacctggtacaagcgcctttacaggag
ttgatagacctgaatttaatcgtatacttgatatgtgtaggaacagaga
aatgaatatgataattgttcattatatttctaggttgagtagggaagaa
cctcttgatattattccagtagttacggagcttcttcgattgggagtga
caatagtatcagttaatgaaggtacctttagaccaggtgaaatgatgga
tttgatacacttaataatgaggttacaagcctcacacgatgaaagtaaa
aataaatctgtagctgtgtctaatgctaaggaacttgcaaagagattgg
gaggacatacaggctcaacaccttacggatttgatactgttgaagagat
ggtaccaaatccagaagatggtggaaaattagttgctattagaaggctt
gtacccagcgctcatacttgggaaggagctcatggatctgagggagcag
taataagatgggcttggcaagaaataaaaacacatagagatacaccgtt
taaaggggggggtgctggtagctttcatcctggttctttaaatggatta
tgtgaaagactttatagagataaggtaccaacaagaggaactctagtgg
gaaaaaaacgcgccggttccgattgggatccaggagtactaaaaagagt
gttatcagaccctagaattgcaggctatcaggccgatatagcttataaa
gtaagagcagatggaagtcgaggtggattttctcattacaaaataagaa
gggaccctgtaactatggagcctctgactcttcccggctttgaaccata
tataccacctgcagagtggtgggaattacaggagtggcttcaaggaagg
ggtagaggcaagggacaatatagaggacagagcctcttatcagcaatgg
atgttttatattgctatggatcaggacagttggatccagaaactggtta
ctccaacggttcgactatggcaggaaatgttagagaaggggatcaagca
cataaaagtagttatgcatgtaaatgtccaagaagagttcatgatggga
gttcttgctcaataacaatgcataatttagatccttatattgttggagc
aatatttgcaagaattaccgctttcgatccagctgaccctgatgattta
gaaggagacactgcagcgttaatgtatgaagctgctagaaggtggggtg
ctactcatgaaagaccagaactcaaaggtcaacgttctgaacttatggc
tcaaagagcagatgctgtaaaagcacttgaagaattatatgaagataaa
agaaatggaggttacagaagtgctatgggaagacgggcttttctagagg
aggaagcagcgctaactttaagaatggaaggggccgaggaaagactgag
acaattagatgctgctgatagtccagtcttaccaattggagaatggtta
ggtgaccgtggatcagatccaactggacctggctcatggtgggcacttg
ctccgctggaggatcgacgtgcatttgttagattatttgtagatagaat
agaagtaataaagcttcctaaaggtgttcaaagacctgggagagttcct
cctatagctgatcgggttagaatccactgggcaaagcctaaggtggaag
aggaaacagaaccagagacacttaacggtttcacggcagcagcataa

In some embodiments, the TG1 recombinase comprises an amino acid sequence of SEQ ID NO: 6. In some embodiments, the TG1 recombinase homolog comprises an amino acid sequence having at least 90% sequence identity to the amino acid sequence shown in SEQ ID NO: 6. In some embodiments, the TG1 recombinase homolog comprises an amino acid sequence having at least 91% sequence identity to the amino acid sequence shown in SEQ ID NO: 6. In some embodiments, the TG1 recombinase homolog comprises an amino acid sequence having at least 92% sequence identity to the amino acid sequence shown in SEQ ID NO: 6. In some embodiments, the TG1 recombinase homolog comprises an amino acid sequence having at least 93% sequence identity to the amino acid sequence shown in SEQ ID NO: 6. In some embodiments, the TG1 recombinase homolog comprises an amino acid sequence having at least 94% sequence identity to the amino acid sequence shown in SEQ ID NO: 6. In some embodiments, the TG1 recombinase homolog comprises an amino acid sequence having at least 95% sequence identity to the amino acid sequence shown in SEQ ID NO: 6. In some embodiments, the TG1 recombinase homolog comprises an amino acid sequence having at least 96% sequence identity to the amino acid sequence shown in SEQ ID NO: 6. In some embodiments, the TG1 recombinase homolog comprises an amino acid sequence having at least 97% sequence identity to the amino acid sequence shown in SEQ ID NO: 6. In some embodiments, the TG1 recombinase homolog comprises an amino acid sequence having at least 98% sequence identity to the amino acid sequence shown in SEQ ID NO: 6. In some embodiments, the TG1 recombinase homolog comprises an amino acid sequence having at least 99% sequence identity to the amino acid sequence shown in SEQ ID NO: 6.

SEQ ID NO: 6
MVILAGGYDRQSAERENSSTASPATQRAANRGKAEALAKEYARDGVEVK
WLGHFSEAPGTSAFTGVDRPEFNRILDMCRNREMNMIIVHYISRLSREE
PLDIIPVVTELLRLGVTIVSVNEGTFRPGEMMDLIHLIMRLQASHDESK
NKSVAVSNAKELAKRLGGHTGSTPYGFDTVEEMVPNPEDGGKLVAIRRL
VPSAHTWEGAHGSEGAVIRWAWQEIKTHRDTPFKGGGAGSFHPGSLNGL
CERLYRDKVPTRGTLVGKKRAGSDWDPGVLKRVLSDPRIAGYQADIAYK
VRADGSRGGFSHYKIRRDPVTMEPLTLPGFEPYIPPAEWWELQEWLQGR
GRGKGQYRGQSLLSAMDVLYCYGSGQLDPETGYSNGSTMAGNVREGDQA
HKSSYACKCPRRVHDGSSCSITMHNLDPYIVGAIFARITAFDPADPDDL
EGDTAALMYEAARRWGATHERPELKGQRSELMAQRADAVKALEELYEDK
RNGGYRSAMGRRAFLEEEAALTLRMEGAEERLRQLDAADSPVLPIGEWL
GDRGSDPTGPGSWWALAPLEDRRAFVRLFVDRIEVIKLPKGVQRPGRVP
PIADRVRIHWAKPKVEEETEPETLNGFTAAA

In some embodiments, the thermophile bacterium cell is any bacterial cell that can grow and thrive at thermophilic temperatures. In some embodiments, the thermophile bacterium cell is from the genus of Bacillus, Geobacillus, Paenebacillus, Clostridium, Anaerocellum, Caldicellulosiruptor, Thermus, Pyrococcus, Thermococcus, Thermoanaerobacter, Thermoanaerobacterium, Herbinix, Acetivibrio, or Acidothermus. In some embodiments, the bacterium cell is a Clostridium thermocellum, Geobacillus thermoglucosidasius, or Bacillus licheniformis.

In some embodiments, the thermophile bacterial cell comprises multiple att sites in its chromosome, and the system comprises another cargo plasmid comprising another corresponding att site. In some embodiments, the another corresponding att site on the another cargo plasmid is an att site recognized by a different recombinase than the att site on the previous cargo plasmid. For example, the bacterial cell chromosome comprises multiple attB sites, each site being specific for a different recombinase. A cargo plasmid has a cognate attP site specific for a Y412MC61 recombinase (referred to as “Y412MC61 attP”). The Y412MC61 attB site in the bacterial chromosome works with the Y412MC61 attP site on the cargo plasmid to integrate the cargo plasmid into the bacterial chromosome. Then, for example, an additional cargo plasmid having a cognate attP site for the BXB1 recombinase is used so that the BXB1 attB site in the bacterial chromosome works with the cognate BXB1 attP site in the additional cargo plasmid to insert the additional cargo plasmid into the bacterial chromosome.

### Methods of Thermostable Insertion

One aspect of the disclosure is directed to a method for the thermostable insertion of a heterologous DNA into a chromosome of an organism, the method comprising:
a) transfecting a cargo plasmid into a genetically engineered thermophile bacterial cell comprising at least one att site in its chromosome wherein the att site is one member of a pair of attB and attP recombination sites,
wherein the thermophile bacterial cell expresses a thermophilic site-specific recombinase that recognizes the pair of attB and attP recombination sites;
wherein the cargo plasmid comprising a corresponding att recombination site, wherein the corresponding att recombination site is the other member of the pair of attB and attP recombination sites;
wherein the cargo plasmid comprises the heterologous DNA;
wherein the cargo plasmid is capable of insertion into the chromosome of the thermophile bacterial cell at the at least one att site via recombination with the at least one corresponding att site on the cargo plasmid; and
wherein the recombination results in the formation of an attL site and an attR site, wherein the attL and attR sites are not substrates for further recombination.
wherein the insertion is a site-specific recombination at a temperature at or above 50° C. and is thermostable; and
b) selecting a thermophile bacterial cell wherein the heterologous DNA is inserted into the chromosome of the thermophile bacterial cell.

In some embodiments, the expression of the thermophilic site-specific recombinase is achieved by transfecting, in step (a), a helper plasmid into the thermophile bacterial cell, wherein the helper plasmid comprises a nucleic acid sequence encoding the site-specific recombinase.

In some embodiments, the method further comprises a step c) culturing the selected bacterial cell under conditions suitable for growth and replication. In some embodiments, the culturing comprises culturing at a temperature at or above 55° C.

In some embodiments, the cargo plasmid further comprises a selectable marker gene. Selectable marker genes are genes that are added to cells to give them a trait that makes them easy to identify and select. Selectable marker genes are well-known in the art and include, but are not limited to, antibiotic resistance genes and visual reporter genes. In some embodiments, the selection of a thermophilic bacterial cell wherein the heterologous DNA is inserted into the chromosome of the thermophile bacterial cell of step (b) is based on the selectable marker.

In some embodiments, the selectable marker gene is flanked by another pair of attB and attP recombination sites recognized by another thermophilic site-specific recombinase. In some embodiments, the selectable marker gene is removed from the selected thermophile bacterial cell via recombination mediated by the another thermophilic site-specific recombinase. In some embodiments, the another thermophilic site-specific recombinase is expressed from a helper plasmid introduced into the selected thermophile bacterial cell. In some embodiments, the thermophilic site-specific recombinase and the another thermophilic site-specific recombinase are a serine recombinase or a tyrosine recombinase. For example, the att pair used for integration of the cargo plasmid into the bacterial chromosome is an attBlattP pair specific to a Y412MC61 recombinase, while another pair of att recombination sites is a pair of attBlattP recombination sites which flank the selectable marker gene that is located in the cargo DNA which gets inserted into the bacterial chromosome. The additional pair of att recombination sites is a pair of attB/attP recombination sites that are specific to the BXB1 recombinase. As such, the additional pair of att recombination sites flanking the selectable marker gene allows for the selectable marker gene to be excised from the bacterial chromosome through the use of BXB1 recombinase once the selectable marker is no longer needed. In some embodiments, the serine recombinase is Y412MC61, BXB1, or TG1.

In some embodiments of the method for the thermostable insertion of a heterologous DNA into a chromosome of an organism, the thermophile bacterial cell comprises multiple att sites in its chromosome, wherein the method comprises transfecting into the thermophile bacterial cell another cargo plasmid comprising another corresponding att site and another heterologous DNA, and selecting a thermophile bacterial cell in which the another heterologous DNA is also integrated in the chromosome. In some embodiments, the multiple att sites are used for serial insertion, i.e. multiple insertions that occur one after another. For example, the bacterial cell chromosome comprises multiple attB sites, each site being specific for a different recombinase. A cargo plasmid has a cognate attP site specific for the Y412MC61 recombinase, so the attB site in the bacterial chromosome specific for the Y412MC61 recombinase works with the Y412MC61 attP site on the cargo plasmid to integrate the cargo plasmid into the bacterial chromosome. Then, an additional cargo plasmid having a cognate attP site for the BXB1 recombinase so that the attB site in the bacterial chromosome specific for the BXB1 recombinase works with the cognate BXB1 att site in the additional cargo plasmid to insert the additional cargo plasmid into the bacterial chromosome.

### Bacterial Cell Product of Disclosed Insertion Methods

Another aspect of the disclosure is directed to a thermophile bacterial cell made through the method for the thermostable insertion of a heterologous DNA into a chromosome of an organism as disclosed herein.

In some embodiments, the thermophile bacterial cell produced by the disclosed methods comprises the DNA inserted, flanked by an attL scar site and an attR scar site.

In some embodiments, the product thermophile bacterial cell further comprises a selectable marker gene in its genome.

Another aspect of the disclosure is directed to a thermophile bacterial cell, comprising a heterologous DNA inserted in the chromosome of the bacterial cell, wherein the heterologous DNA is flanked by an attL site and an attR site. The heterologous DNA is a DNA that is not native to the thermophile bacterial cell, i.e. a foreign DNA fragment. When the heterologous DNA is flanked by an attL site and an attR site, there is an attL site on one side and an attR site on the other side.

### Removal of DNA

Some aspects of the present disclosure are directed to the removal of DNA from the genome of a thermophile bacterial cell. While removal of genes and methods for the removal of genes is known in the art, conventional gene removal methods permanently remove the targeted gene. For example, a genetic engineered knockout of a gene removes that gene without the possibility of expression later. However, there is benefit to controlling the removal of the target gene and only removing the gene when desired. As such, certain aspects of the present disclosure are directed to the controlled removal of DNA from the genome of a thermophilic bacterial cell.

### Thermophile Bacterial Cell Engineered for Removal of DNA

One aspect of the disclosure is directed to a thermophile bacterial cell, comprising in its chromosome, a DNA flanked by a pair of attB and attP recombination sites.

In some embodiments, the DNA is a DNA native to the thermophile bacterial cell with an attB site on one side of the DNA sequence and an attP site on the other side of the DNA sequence. In some embodiments, the DNA is a DNA heterologous to the thermophile bacterial cell with an attB site on one side of the DNA sequence and an attP site on the other side of the DNA sequence. The flanking of a DNA with a pair of attB and attP recombination sites allows for the controlled removal of the DNA.

A pair of attB and attP can be placed in the chromosome to flank a DNA desired to be removed by utilizing any suitable methodology. Methods of preparation are known in the art, any of which can be used. In some embodiments, the att site is inserted through homologous recombination, wherein a plasmid is transformed into the bacterial chromosome, the transformation is confirmed through PCR, and isolates with confirmed genomic integration were selected.

### Systems for the Removal of DNA

Some aspects of the disclosure are directed to a system for excising DNA from the chromosome of an organism, the system comprising:
the thermophile bacterial cell comprising in its chromosome, a DNA flanked by a pair of attB and attP recombination sites; and
a nucleic acid encoding a site-specific recombinase that performs recombination between the attB and attP recombination sites; and
wherein the recombination results in the formation of an attL site and an attR site, wherein the attL and attR sites are not substrates for further recombination, and the DNA is removed from the chromosome.

In some embodiments of the disclosure, the thermophilic site-specific recombinase is a serine recombinase or a tyrosine recombinase. In some embodiments, the serine recombinase is selected from Y412MC61, BXB1, and TG1, and homologs thereof. In some embodiments, the excised DNA is a DNA native to the thermophile bacterial cell. In some embodiments, the excised DNA is a DNA heterologous to the thermophile bacterial cell.

### Methods for the Removal of DNA

One aspect of the disclosure is directed to a method for excising DNA from the chromosome of an organism, the method comprising:
introducing a pair of attB and attP recombination sites into a thermophile bacterial cell, such that the sites flank a DNA on the chromosome; and
expressing a thermophilic site-specific recombinase that performs recombination between the attB and attP recombination sites; and
wherein the recombination results in the formation of an attL site and an attR site, wherein the attL and attR sites are not substrates for further recombination, and the DNA is removed from the chromosome.

One aspect of the disclosure is directed to a method for excising DNA from the chromosome of an organism, the method comprising:
providing the thermophile bacterial cell, comprising in its chromosome, a DNA flanked by a pair of attB and attP recombination site; and
expressing a thermophilic site-specific recombinase that performs recombination between the attB and attP recombination sites; and
wherein the recombination results in the formation of an attL site and an attR site, wherein the attL and attR sites are not substrates for further recombination, and the DNA is removed from the chromosome.

In some embodiments, the thermophilic site-specific recombinase is expressed from a plasmid introduced into the thermophile bacterial cell. In some embodiments, the thermophilic site-specific recombinase is a serine recombinase or a tyrosine recombinase. In some embodiments, the serine recombinase is selected from Y412MC61, BXB1, and TG1, and homologs thereof. In some embodiments, the excised DNA is a DNA native to the thermophile bacterial cell. In some embodiments, the excised DNA is a DNA heterologous to the thermophile bacterial cell.

### Bacterial Cell Product of Disclosed Excision Methods

One aspect of the disclosure is directed to a thermophile bacterial cell, comprising in its chromosome, a DNA flanked by a pair of attB and attP recombination sites. In some embodiments, the DNA is a DNA native to the thermophile bacterial cell. In some embodiments, the DNA is a DNA heterologous to the thermophile bacterial cell. Another aspect of the disclosure is directed to a thermophile bacterial cell made through the method for excising DNA from the chromosome of an organism disclosed herein. In some embodiments, the thermophile bacterial cell comprises in its chromosome, an attL scar site and an attR scar site at the site of recombination, wherein the attL and attR sites are not substrates for further recombination, and the target DNA is removed from the chromosome. The gene encoded by the DNA sequence is silenced due to the removal of the sequence.

### EXAMPLES

The following examples are set forth as being representative of the present disclosure. These examples are not to be construed as limiting the scope of the present disclosure as these and other equivalent embodiments will be apparent in view of the present disclosure, figures and accompanying claims.

### Example 1: Development of Thermophilic SAGE (tSAGE) Recombination System for C. thermocellum

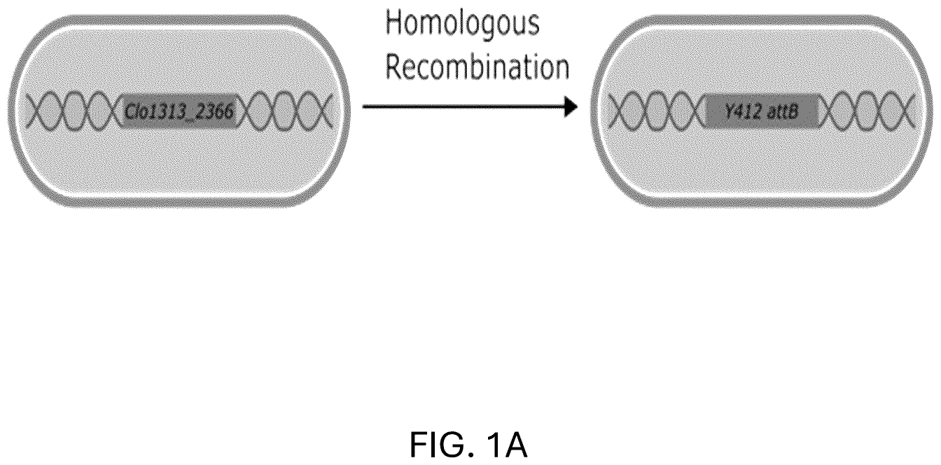

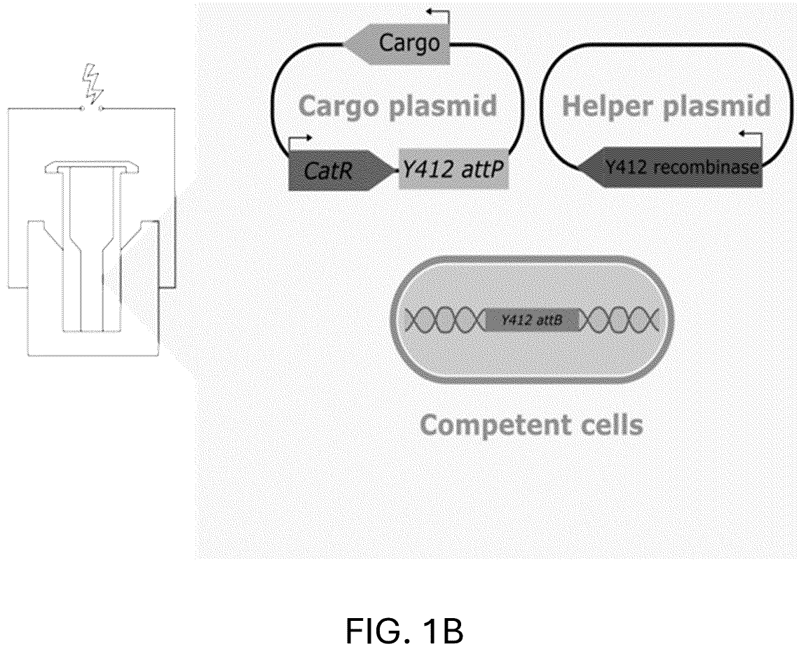

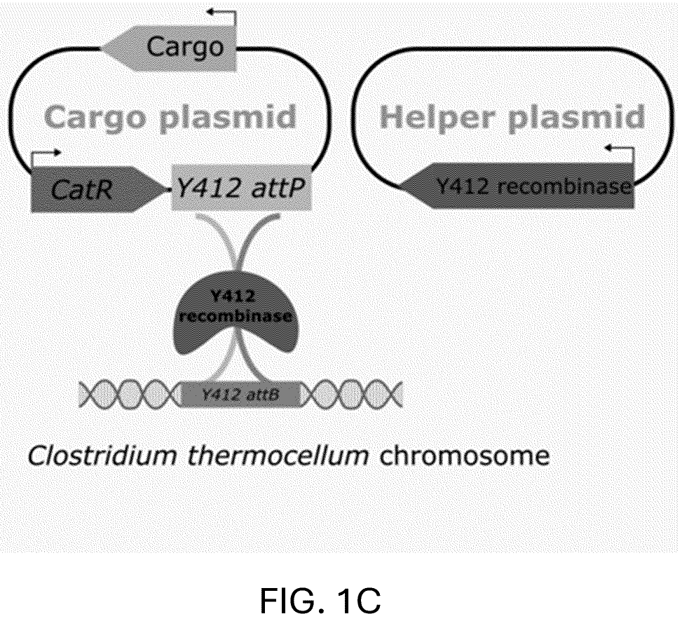

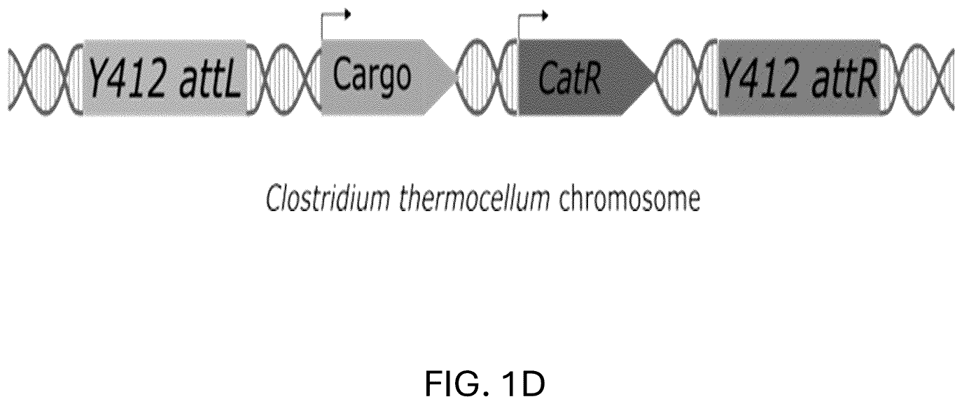

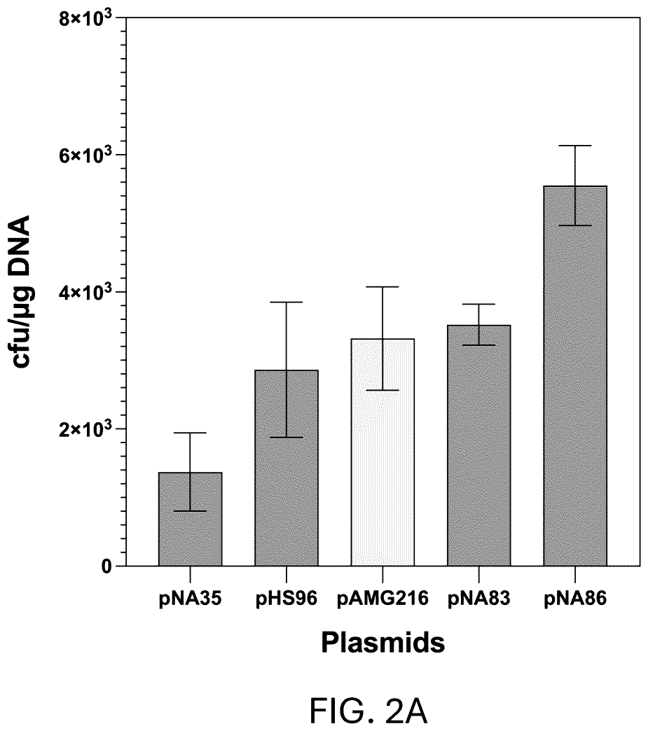

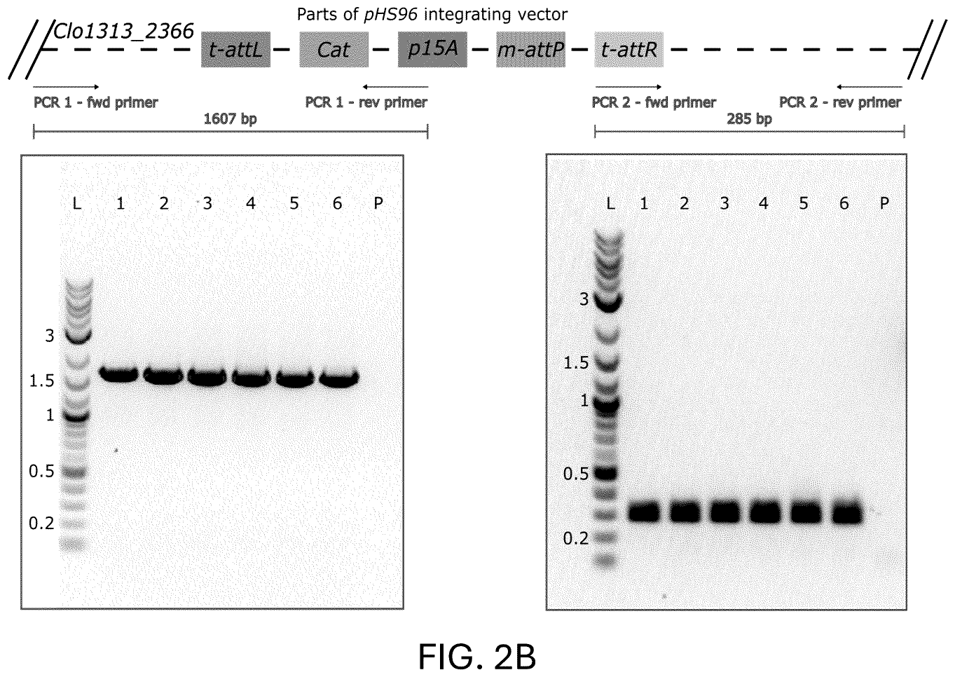

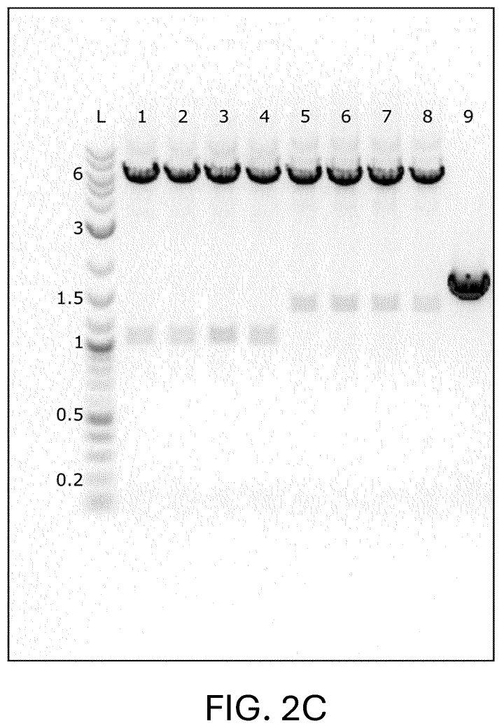

A strain of C. thermocellum was constructed that contains a “landing pad” of 13 orthogonal attB sites inserted into the Clo1313_2366 locus of strain LL1299, resulting in strain AG8235. The functionality and efficiency of the Y412MC61 recombinase was first tested by co-transforming this strain with two plasmids: 1) pHS96, which contains the 16 corresponding attP sites, a cat gene that confers thiamphenicol resistance driven by the GAPDH promoter, a second cat gene that is an artifact of a previous cloning strategy, and the E. coli p15A origin of replication and 2) pNA42, which expresses the Y412MC61 recombinase from a Clo1313_2638 promoter (the helper plasmid) (FIG. 1). The transformation efficiency was equivalent between tSAGE-based chromosomal integration and transformation of a replicating control plasmid: 2.86×10³ CFU/μg for integration of pHS96 and 3.32×10³ CFU/μg of the replicating pAMG216 (FIG. 2A). PCR testing of the resultant colonies confirmed integration of the 4.2 kb pHS96 at the Y412MC61 attB site of AG8235 with all tested colonies having inserted properly (FIG. 2B). The Y412MC61 recombinase is thus a thermostable enzyme capable of efficient insertion of DNA into the chromosome in thermophilic organisms like C. thermocellum.

Having more than one thermophilic recombinase could be useful for various applications, including removal of the selectable marker from used for SAGE insertion. While the remaining recombinases are from mesophilic organisms, some may be thermostable by chance. Therefore, each of the other recombinases were tested using the helper plasmids pNA56-G, and pNA58-G through pNA66-G, each of which express a different mesophilic serine recombinase expressed using the P2638 promoter (Table 2). Each helper plasmid was co-transformed with then poly-attP plasmid. Of these, only the TG1 and BXB1 recombinases enabled recombination between their cognate attP and attB sites, with a transformation efficiency of 100 and 40 CFU/μg of integration vector, respectively. The transformation efficiency of the control vector (pAMG216) using the same competent cells was 4×10³ CFU/μg, indicating that each can function under thermophilic conditions, albeit less well than Y412MC61.

### Example 2: Reporter Screening Using tSAGE

We used tSAGE to characterize chromosomally encoded genetic parts. Promoter characterization studies often use a reporter gene and many reporter genes are known in the art. Previous efforts focused on characterizing promoters on replicating plasmids, and here, we wanted to do the same from the chromosome because most metabolic engineering efforts focus on chromosomally inserted heterologous pathways. Therefore, multiple potential reporter genes were first tested for use in C. thermocellum. Many of the most widely used reporters require O₂ to become fluorescent, such as green fluorescent protein (GFP) and its color, stability, and brightness variants. However, these proteins can be synthesized anaerobically and then exposed to O₂ to allow protein maturation and create the fluorophore. Anaerobically active flavin-based fluorescent proteins (FbFp) have also been described, though they are typically not as bright as GFP. tSAGE was used to integrate nine reporters into the genome of C. thermocellum strain AG8235, including two variants of the FbFp gene from Chloroflexus aggregans (cagFbFP-V1 and cagFbFP-V2, the wildtype gene for YNP3-FbFp and its reportedly brighter variant YNP3Y116F-FbFp, the FbFp from Meiothermus ruber (mrFbFp), and two versions each of mKATE and superfolder GFP (sfGFP), with and without codon optimized. In all these plasmids, the reporter gene was driven by the PClo1313_1194 promoter from C. thermocellum DSM1313. This promoter was previously shown to be a strong promoter in C. thermocellum.

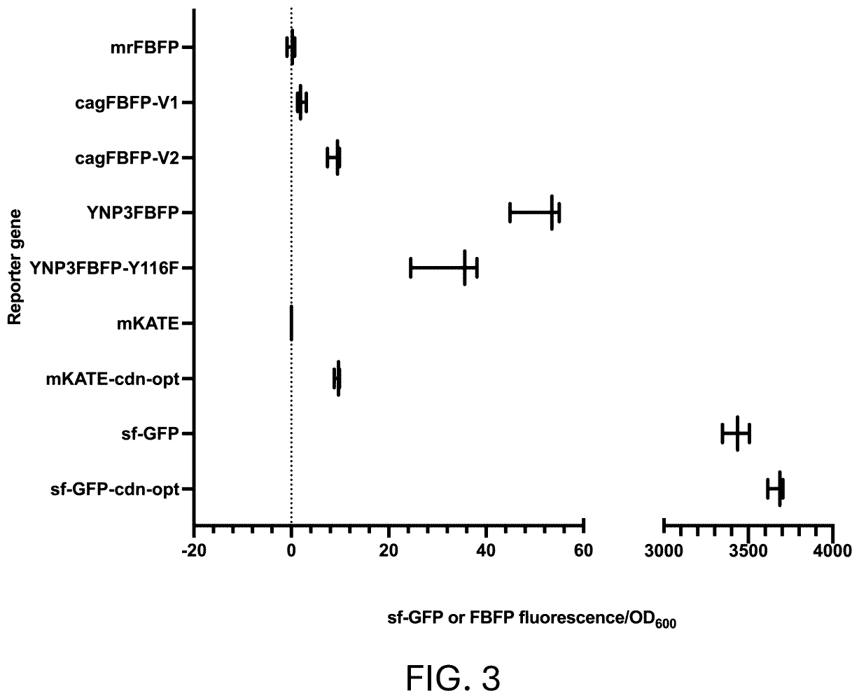

Wildtype YNP3 FbFp was the brightest among the flavin-based fluorescent proteins (50.1±6.5 fluorescent units), while the FbFp from Chloroflexus aggregans was found to be the dimmest (1.8±0.76 fluorescent units). Expression of mKATE did not result in any detectable fluorescence in C. thermocellum. The two sfGFP variants were found to be the brightest reporters in C. thermocellum, being ˜60-fold brighter than the brightest FbFp (FIG. 3). Subsequently, sfGFP was the chosen reporter for screening promoters in C. thermocellum.

### Example 3: Promoter Screening Using t-SAGE

Rational strain engineering typically requires an array of well-characterized low-, mid-, and high-strength constitutive promoters. Olsen et al. previously tested a replicating plasmid-based library of homologous promoters using thermostable beta-galactosidase and alcohol dehydrogenase genes as reporters, and this work was extended by characterizing chromosomally inserted promoters. When choosing promoters, native/homologous promoters can have higher expression levels, while heterologous promoters can be more stable by not being substrates for homologous recombination with the native locus. Therefore, tSAGE was used to integrate 15 native C. thermocellum promoters and 31 heterologous promoters from different thermophiles fused to sfGFP into the AG8235 chromosome. Each of the integrating vectors that were used in this promoter library (Table 2) have the same backbone with a Y412MC61 attP site, sfGFP, a colEl origin for propagation in E. coli, and a thiamphenicol resistance gene driven by the C. thermocellum P_{gapD} promoter, and they were inserted using helper plasmid pNA42, as above.

Among the homologous promoters tested, promoters of genes clo1313_1194 (AG9469), clo1313_3011 (2 versions-AG9462, AG9178), and clo1313_2638 (AG9176) had the highest sfGFP expression of 3471, 1747, 1592 and 1494 fluorescence units, respectively. In comparison, the homologous gapD promoter from C. thermocellum (gene locus ID: clo1313_2095, 2 versions-AG9170 and AG9171), a commonly used promoter for strong expression in this organism, had sfGFP expression levels of 401±26 (version 1) and 317±17 (version 2) fluorescence units.

BLAST-P was used to identify homologs of the top three C. thermocellum genes in the closely related thermophilic anaerobe Acetivibrio clariflavus DSM 19732 and the more distantly related thermophilic facultative anaerobe Geobacillus thermodenitrificans NG80-2, with the upstream region of the top hits being used as additional promoters in the library (Table 1). Additionally, promoters of the lactate dehydrogenase gene from Geobacillus stearothermophilus, the ribosomal rRNA gene from Geobacillus thermoglucosidasius, and the historically used promoters from Thermoanaerobacterium saccharolyticum JW/SL-YS485 (of genes Tsac_0068, Tsac_0530, and Tsac_0046) were also tested.

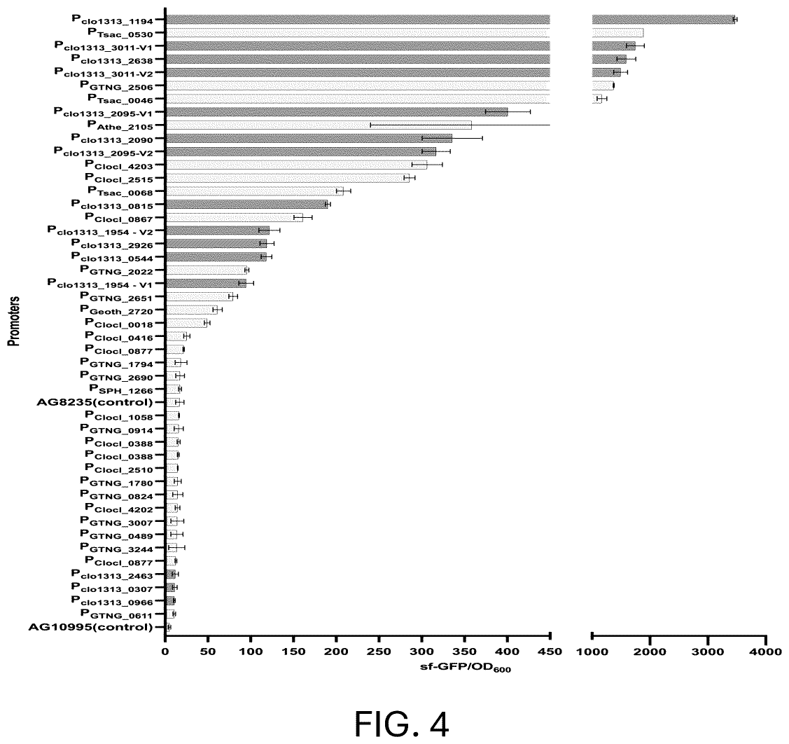

The promoter of the gene Tsac_0530 from T. saccharolyticum JW/SL-YS485 was the strongest among the heterologous promoters with sfGFP expression of 1890 fluorescence units. The promoter of the gene gtng_2506 from G. thermodenitrificans NG80-2, an ortholog to the clo1313_2638 gene from C. thermocellum, and the promoter of gene Tsac_0046 from T. saccharolyticum JW/SL-YS485 were also strong heterologous promoters. The promoter of the gene Athe_2105 from C. bescii was a similar strength to the native, commonly used promoter from C. thermocellum, P_{gapD}. Promoters of genes Clocl_2515 and Clocl_4203 from A. clariflavus DSM 19732 also had similar strength to the C. thermocellum, P_{gapD} promoter. The cat promoter, a promoter that drives the chloramphenicol acetyltransferase gene from different strains of Streptococcus pneumoniae, which has previously been used in C. thermocellum, had very low expression in C. thermocellum. Taken together, a library of promoters was built that contains strong, mid-level, and weak promoters from homologous and heterologous sources (FIG. 4) for use in strain engineering in C. thermocellum.

### Example 4: Inducible Promoter and Riboswitch Screening Using t-SAGE

Inducible promoters and riboswitches are essential tools in synthetic biology that enable time- and dose-dependent gene expression. Therefore, chromosomally encoded, regulated gene expression systems were explored.

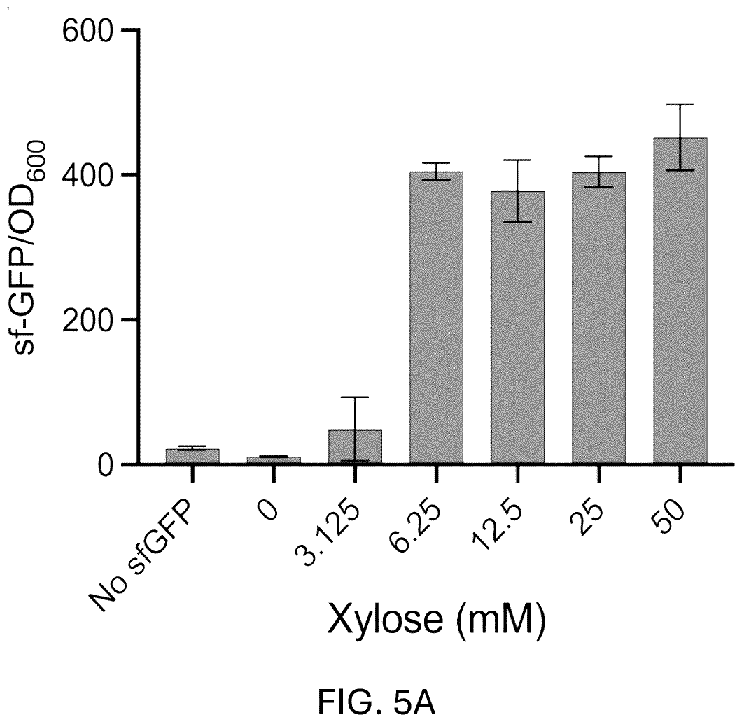

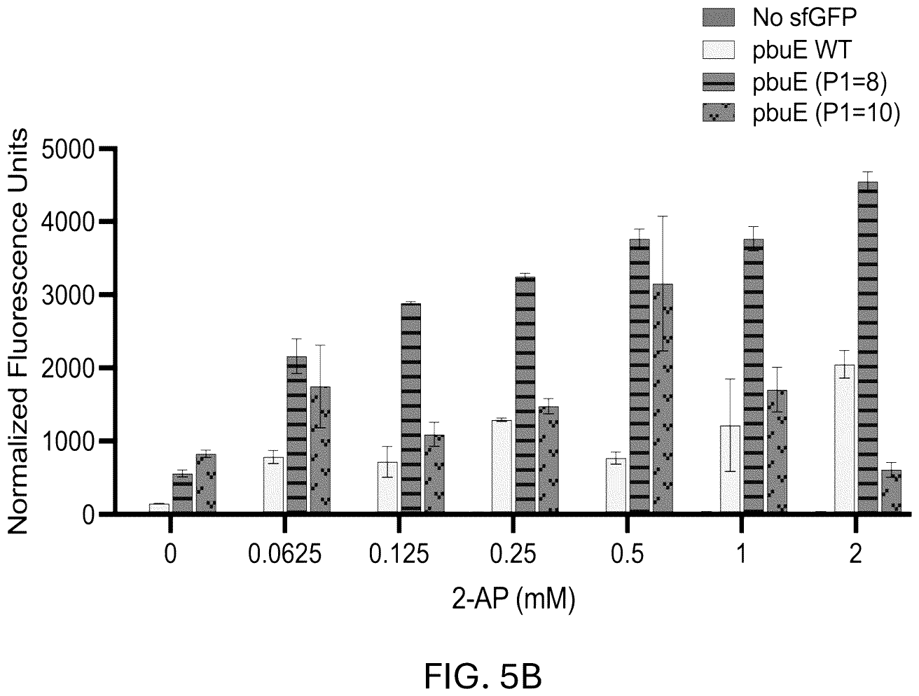

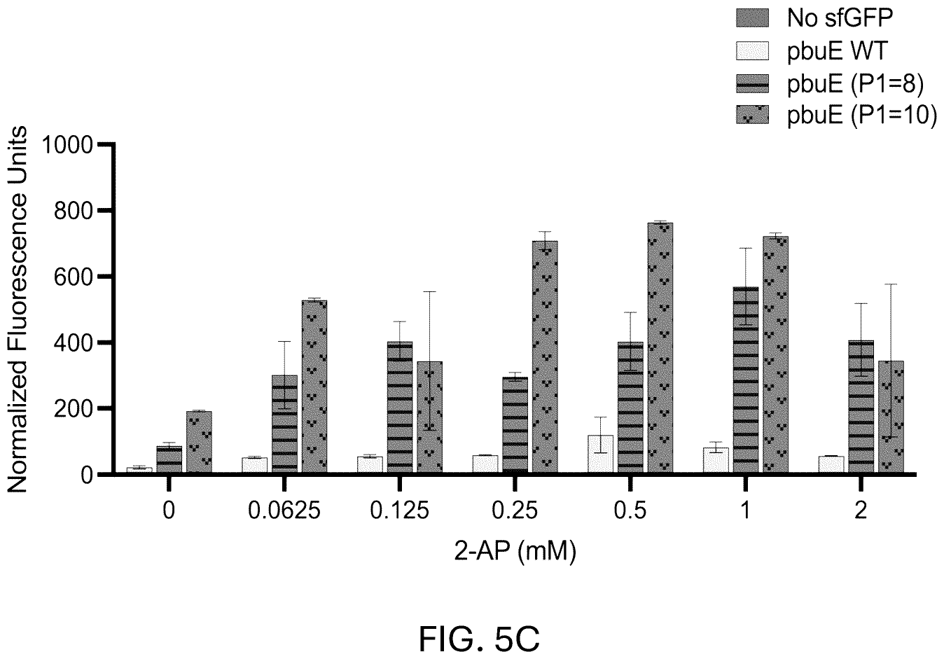

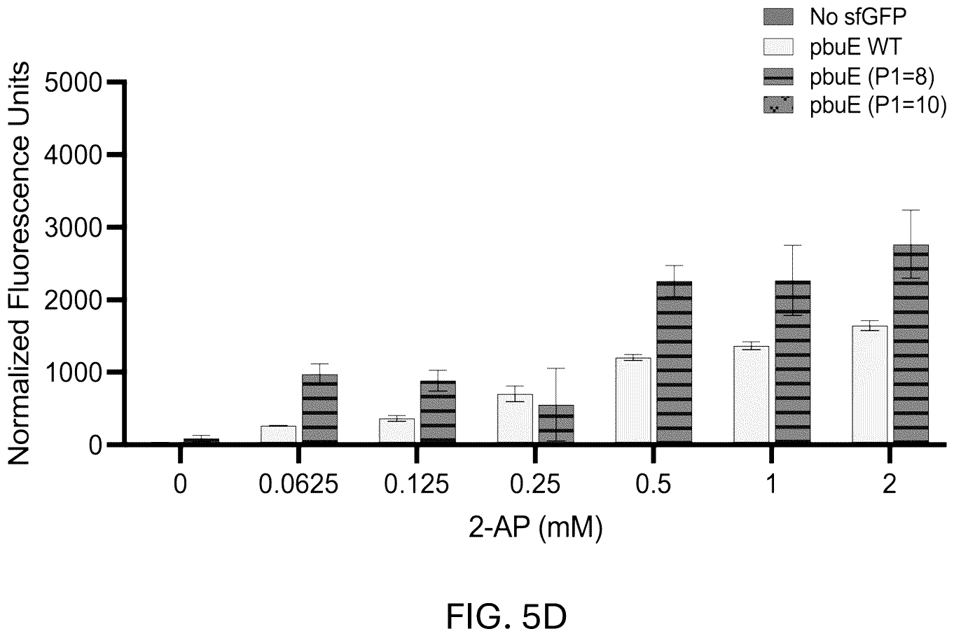

Xylose is a potentially useful inducer molecule because it is not natively metabolized by C. thermocellum, and so it can be used as an orthogonal inducer, but C. thermocellum has also been engineered to utilize xylose, and so it could also be used for expression transiently or specifically during growth on lignocellulose. Therefore, a xylose-inducible expression system was designed based on the system native to C. bescii. In C. thermocellum, this promoter resulted in approximately 40-fold increased sfGFP expression when induced with 6.25 mM or higher concentrations of xylose (FIG. 5A). In the absence of xylose, the expression level is low, indicating tight control of gene expression. The xylose-inducible promoter from Clostridioides difficile, the cumate inducible promoter from Pseudomonas putida, and the arabinose inducible promoter from E. coli were also tested, but they were not inducible in C. thermocellum (data not shown).

Riboswitches represent a mechanism to control gene expression post-transcriptionally. As described in U.S. Pat. No. 11,198,871 2-aminopurine (2-AP)-inducible pbuE riboswitches identified in Bacillus subtilis function in C. thermocellum strains on replicating plasmids, but the riboswitches showed background expression without the inducer 2-AP. To increase the tightness of the riboswitches and test their activities as genomically integrated single-copy expression cassettes, the wild-type and two mutated pbuE riboswitches fused to sfGFP were integrated into the chromosome using tSAGE. The mutant pbuE riboswitchs included modifications to extend the P1 stem from 5 base pairs to 8 (P1=8) and 10 (P1=10) base pairs to improve the sensitivity and inducibility. When driven by the P_{Clo1313_1194} promoter, all three pbuE riboswitches showed moderate inducibility (approximately 14-, 8-, and 3-fold induction, respectively) but also leakiness when uninduced (FIG. 5B). To reduce leakiness of the pbuE riboswitches, the strong P_{Clo1313_1194} promoter was replaced with medium-strength promoters P_{Tsac_0068} and P_{gapD}. The P_{Tsac_0068} promoter did not decrease the leakiness, but it reduced the inducibility (FIG. 5C). The P_{gapD} promoter improved both the leakiness and the inducibility in the WT and P1=8 pbuE riboswitches, with an approximately 50- and 30-fold difference in expression for induced versus uninduced, respectively (FIG. 5D). The P1=10 riboswitch could not be tested under the control of the PGAPDH promoter due to multiple unsuccessful attempts at transformation of the plasmid into C. thermocellum. In addition to the pbuE riboswitches, two fluoride-inducible riboswitches were also tested, but neither of them showed induction of gene expression (data not shown).

### Example 5: Ribosome Binding Site (RBS) Library Screening Using t-SAGE

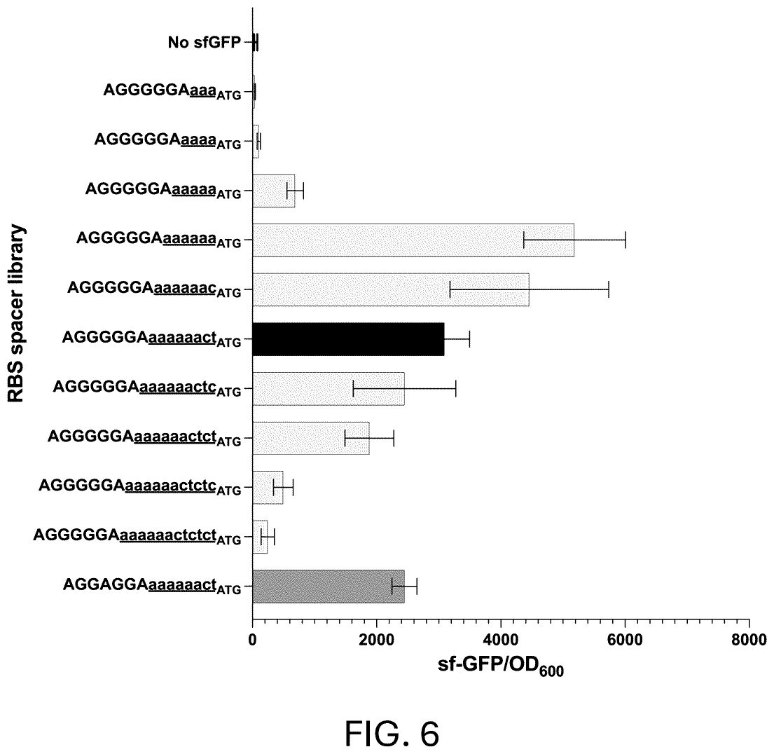

The PClo1313_1194 promoter, the most highly expressed homologous promoter in our library, has an RBS and spacer sequence reading ‘AGGGGGAAAAAAACT’ (SEQ ID NO: 25) before the ATG start codon (RBS underlined). To find the best distance between the RBS and the start codon in C. thermocellum, a library was created in which the distance between the RBS and the start codon was varied from 3 to 12 bases. The consensus RBS was also tested by changing the RBS of P_{Clo1313_1194} to ‘AGGAGGAAAAAAACT’ (SEQ ID NO: 26). The resulting library was genomically integrated into the chromosome of C. thermocellum using tSAGE and the sfGFP fluorescence was measured (FIG. 6). The nucleotide distance of 6-7 bases between the RBS and the start codon was found to result in the highest gene expression from PClo1313_1194. Also, the consensus RBS (AGGAGGA) performed similarly to the native RBS.

### Example 6: Excision of Plasmid Backbone Using BXB1 and TG1 Recombinases

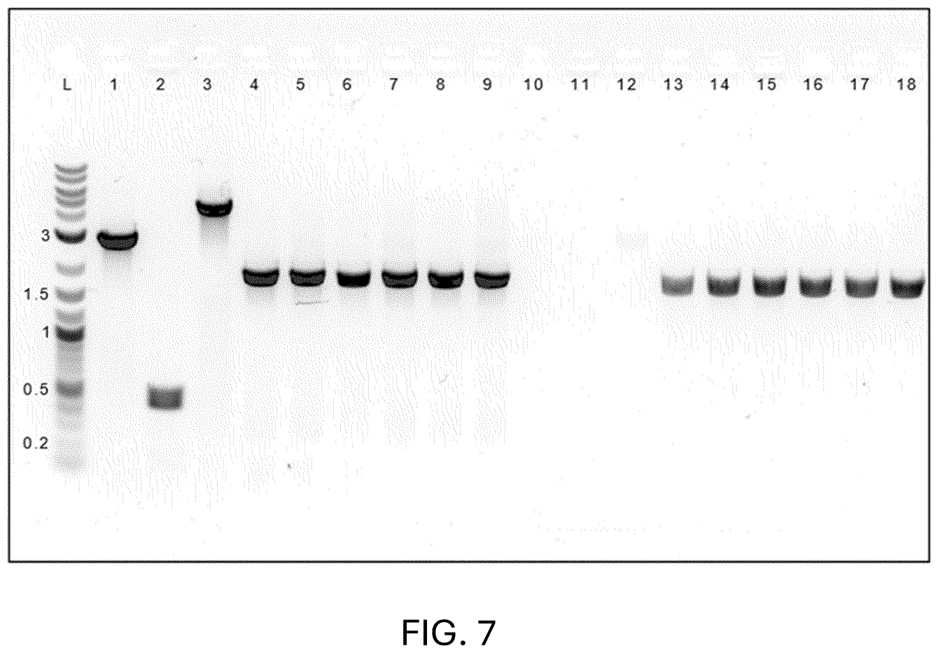

As described above, tSAGE integrates the complete cargo plasmid irreversibly into the chromosome of C. thermocellum, leaving the antibiotic resistance gene on the chromosome. After the genetic cargo of interest gets inserted into the chromosome, removal of the E. coli origin of replication and the thiamphenicol resistance gene would allow further genetic modifications. With the discovery that TG1 and BXB 1 recombinases function in C. thermocellum, they were tested for use to remove the backbone of the integrated plasmid (FIG. 1B). Since the landing pad of AG8235 has 13 attB sites, including attB sites specific to BXB1 and TG1, homologous recombination was used to place the single Y412MC61 attB at the Clo1313_2366 locus of strain LL1299. The resulting strain, AG10995, was used to test backbone excision using BXB1 recombinase. A BXB1 attB site and attP site was introduced on either side of the genetic cargo of pNA28 to make pNA122, which was inserted into the chromosome of AG10995 by tSAGE. The resulting strain, AG11004, was used to test the efficiency of backbone excision using BXB1 recombinase. AG11004 was transformed with pNA56G, which expresses the Bxb1 recombinase, and plated on neomycin to select for the plasmid. The resulting colonies were grown in liquid culture overnight and screened by PCR to test for excision. Nine out of ten colonies had undergone backbone excision (FIG. 7), and those strains were each thiamphenicol sensitive. Thus, BXB1 recombinase efficiently excised the backbone and rendered a final strain with a small 50 bp recombination scar and the genetic cargo. Because the pNW33N origin of replication is unstable in C. thermocellum, growth in the absence of neomycin resulted in loss of the BXB1 recombinase plasmid, producing a strain that can be used for further strain engineering.

### Example 7: General Materials and Methods

### Strains and Culturing Conditions

LL1299, a Clostridium thermocellum strain in which a restriction enzyme (Clo1313_0478) was deleted, was the parent strain used throughout this study. AG8235 has a poly-attB landing pad (13 attB sites including the Y412MC61 attB site) replacing the Clo1313_2366 locus, while AG10995 has only the Geobacillus Y412MC61 attB site at the Clo1313_2366 locus. C. thermocellum strains were grown inside a Coy Labs anaerobic chamber using a gas mix of 85% N2, 10% CO2, and 5% H2 on rich CTFUD medium supplemented with 15 μg/mL thiamphenicol (Tm15) when needed to select for plasmids. Plasmids were constructed and maintained in E. coli Top10 Δdcm (strain AG583) grown in LB (Miller) supplemented with 25 μg/mL chloramphenicol with shaking at 37° C.

The attB landing pads were genomically integrated using the homologous recombination method detailed below. Both AG8235 and AG10995 were thiamphenicol sensitive and were cultured in liquid CTFUD. After transformation with the reporter, promoter, riboswitch, and RBS libraries, the strains with the genomically integrated cargo plasmids were plated on CTFUD-agar with 15 μg/mL of Thiamphenicol. They were grown in liquid CTFUD with 15 μg/mL of thiamphenicol overnight for the sf-GFP or FbFp assay. AG11004 were derived after genomic integration of pNA122 at the Y412MC61 attB site of AG10995. AG11004 was thiamphenicol-resistant and was selected on and grown in CTFUD (liquid or agar solidified) with 15 μg/mL of thiamphenicol. After transforming pNA56G into AG11004, the transformants were selected on agar-solidified CTFUD with 300 μg/mL of neomycin. Colonies from the plate were picked into liquid CTFUD with 300 μg/mL of neomycin. Post PCR confirmation for backbone excision, these strains were grown in CTFUD without antibiotics. The strains generated in this study are listed in Table 1.

### Plasmid Construction and Sequencing Confirmations

The plasmids used in this study are listed in Table 2. Computationally designed plasmids were were synthesized by Genscript Inc. All sequencing was confirmed by Sanger sequencing (Genscript Inc.) or Oxford Nanopore Technologies sequencing (Plasmidsaurus).

### Competent Cell Preparation and Transformation

An overnight-grown culture of E. coli was used to inoculate 50 mL of LB in a 250 mL flask to an OD600 of ˜0.05. The culture was then grown for ˜2-3 hours in a shaking incubator at 37° C. to a final optical density at 600 nm (OD600) between 0.5 and 0.7. The cells were centrifuged at 7,000×g in a 4° C. centrifuge, washed thrice with ice-cold 10% glycerol solution, and the pellet was resuspended in 500 μL of 10% glycerol. 30 μL aliquots of electrocompetent cells were either stored at −80° C. for later use or immediately used to transform plasmids. Plasmids were electroporated into E. coli using a Bio-Rad Gene Pulser Xcell set for exponential decay, 25 μF, 200 ohm, 1800 volts, 0.1 cm cuvette. After the electrical pulsing, the cells in the cuvette were resuspended in 950 μL of SOC medium, incubated at 30° C. for 3-4 hours, and plated on LB with appropriate antibiotics (25 μg/mL of chloramphenicol or 50 μg/mL of kanamycin) and incubated at 30° C. overnight. The colonies were confirmed by PCR for successful plasmid transformation. Plasmids were extracted using a Zymo midi prep kit from 50 mL liquid cultures (LB with appropriate antibiotics) using the low copy number plasmid extraction protocol.

C. thermocellum competent cells were made using an already-established protocol (Guss et al., doi: 10.1186/1754-6834-5-30). Briefly, a frozen stock of C. thermocellum stored at −80° C. was used as an inoculum. It was used to inoculate 5 mL of CTFUD (with or without antibiotics), which was grown at 50° C. inside a Coy anaerobic chamber. The 5 mL seed culture was used to inoculate 500 mL CTFUD (with or without appropriate antibiotics) and grown overnight at 50° C. in the anaerobic Coy chamber to an OD600 between 0.5 and 0.7. The culture was then chilled on ice, centrifuged aerobically at 6000 RPM for 15 minutes on a benchtop centrifuge set at 19° C., and washed with ice-cold electroporation buffer (250 mM sucrose and 10% glycerol) three times without resuspending the pellet each time. Then, the washed pellet was resuspended in 100 μL of fresh, sterile electroporation buffer. The cells were either used immediately for transformation or were stored at −80° C. for use later. 30 μL of competent cells and 1 μg of each plasmid were combined in an electroporation cuvette, which was transformed using a square wave pulse (1000V, 1.5 msec, 1 mm cuvette) in a Bio-Rad Gene Pulser Xcell using plasmids. After the electrical pulsing, the cells were resuspended in 1 mL CTFUD (no antibiotic) and recovered at 50° C. overnight inside the anaerobic Coy anaerobic chamber. The recovered culture was plated on CTFUD-agar with appropriate antibiotics. The resulting colonies were picked into CTFUD liquid media with appropriate antibiotics, grown at 50° C., and then confirmed by PCR. The primers used in this work for PCR confirmation of integration are listed in Table 3.

TABLE 3

| Primer | SEQ ID NO: | Sequence |
|---|---|---|
| #10 | 19 | cggtttctttaccagact |
| #11 | 20 | ggtattttacccttgtcgtt |
| #13 | 21 | ctctgctactcgtttttcaatc |
| #14 | 22 | gggaaggatttgaggttagg |
| #49 | 23 | ggcaccaataactgccttaaaga |
| #50 | 24 | cgatgataaacaaaggacggttc |

### Homologous Recombination for Genomic Integration of Genetic Cassettes

For insertion of the attB sites into the chromosome via homologous recombination, after plasmid transformation, colonies typically appeared after 3-4 days and were picked into 5 mL liquid CTFUD with 15 μg/L thiamphenicol (CTFUD-Tm15). The cultures were incubated untill turbid (1 to 3 days) at 50° C. and were then subcultured into 5 mL of fresh CTFUD+Tm15 and incubated at 60° C. to select for genomic integration of the replicating plasmid. Turbid cultures grown at 60° C. were then streak plated on CTFUD+Tm15 agar plates and incubated at 60° C. Single colonies were picked into 5 mL liquid CTFUD+Tm15, grown at 60° C., and genomic integration of the plasmid was confirmed using PCR. Two isolates with genomic integration at the downstream homologous arm and two with genomic integration at the upstream homologous arm were chosen for the next steps. The cultures were sub-cultured in fresh CTFUD (no antibiotic) and grown at 60° C. to allow time for a second recombination event and to lose the plasmid. The cultures were then streaked onto CTFUD-agar with 10 μg/mL FUDR and incubated at 60° C. Individual colonies were picked into liquid CTFUD with 10 μg/mL of FUDR, grown at 60° C., and PCR screened to check for the presence of the genetic cassette. Typically, ˜50% of strains tested reverted to WT while ˜50% of strains contained the genetic cassette from the plasmid transformed.

tSAGE, Screening Mesophilic Recombinases and Backbone Excision Using Mesophilic Recombinases

DNA insertion via tSAGE into the corresponding attB site was accomplished by co-transforming 1 μg of the integrating cargo plasmid and 1 μg of the helper plasmid pNA42 by electroporation into competent cells of C. thermocellum AG8235 or AG10995. After an overnight anaerobic recovery at 50° C., the cells were plated on CTFUD+TM15 agar plates. Colonies typically appear in 2-3 days after incubation at 50° C. Colonies were picked into liquid CTFUD with TM15, and subsequently PCR confirmed.

The mesophilic serine recombinases BXB1, φBT1, φFC1, φRV1, TG1, R4, BL3, PA118, ΦMR11, and q370 were tested in C. thermocellum at 42° C., 48° C., and 50° C. Competent cells of AG8235 were co-transformed with 1 μg of the poly-attP integrating plasmid (pHS96) and 1 μg of each of the eleven-serine recombinase-expressing helper plasmids (Table 2). Following electroporation, the cells were resuspended in CTFUD and recovered overnight at 42° C., 48° C., or 50° C. The cells were then plated on CTFUD+TM15 agar and incubated at 50° C. in the anaerobic Coy chamber. The resulting colonies were picked into liquid CTFUD with TM15 and tested for genomic integration of pHS96 at the clo1313_2366 locus using primers #13, #14, listed in Table 3.

pNA122 is modified pNA28 with BXB1 attB and attP sites flanking the genetic cargo. To test plasmid backbone removal, competent cells of AG11004 (pNA122 genomically integrated using tSAGE) were transformed using electroporation with 1 μg of pNA56G. The cells were resuspended in CTFUD (no antibiotics) and recovered overnight at 50° C. The recovered cells were then plated on CTFUD agar with 300 μg/mL neomycin. Colonies typically appeared after 3-4 days and were picked into liquid CTFUD medium with 300 μg/mL of neomycin. After PCR confirmation for backbone excision, these strains were grown in CTFUD without antibiotics.

### Inducible Gene Expression Assays, FBFP, and Superfolder GFP Assay

To assay the fluorescence of sfGFP and each FbFp, overnight grown cultures were moved to aerobic conditions, washed once with 1×PBS, resuspended in 3 ml of 1×PBS, and then incubated overnight in the dark to enable GFP folding. Where appropriate, PCR-confirmed strains were grown overnight with inducers at the following concentrations. L(+) arabinose (1.56 mM, 3.125 mM, 6.25 mM, 12.5 mM, 25 mM, and 50 mM; EMD Millipore Corp., 178680-100GM), D-(+)-Xylose [43], (3.125, 6.25, 12.5, 25, and 50 mM; Sigma-Aldrich, X1500-500G), Cumate (7.8, 15.6, 31.25, 62.5, 125, 250, and 500 μM; System Biosciences, QM150A-1), 2-aminopurine (0.0625, 0.125, 0.25, 0.5, 1, and 2 mM; Thermo Fisher Scientific, J64919-MD), and sodium fluoride. For sfGFP, the fluorescence was measured with excitation at 488 nm and emission at 510 nm in a BioTek fluorescence plate reader. For the FbFps, five different excitation and emission wavelengths were tested (Ex: 450/9, Em: 480/9; Ex: 464/9, Em: 486/9; Ex: 455/9, Em: 483/9; Ex: 455/9, Em: 491/9; Ex: 465/9, Em: 493/9). The excitation and emission at 465/9 and 493/9 found as the best and used throughout assays. The fluorescence was normalized to an OD600 of 1.0.

TABLE 1

| | Parent | | |
|---|---|---|---|
| Strain ID | strain^{organism} | Genotype/plasmid ID | Selection^{μg/ml} |
| LL1299 | DSM 1313^{ctx} | DSM 1313 Δhpt Δ0478 (laboratory evolved) | — |
| AG8235 | LL1299^{ctx} | DSM 1313 Δhpt Δ0478 | — |
| | | 2366::polyattB/pNA24D | |
| AG10995 | LL1299^{ctx} | DSM 1313 Δhpt Δ0478 | — |
| | | 2366::Y412MC61attB/pNA153 | |
| AG11004 | AG10995^{ctx} | DSM 1313 Δhpt Δ0478 | Tm¹⁵ |
| | | 2366::pNA122/pNA122 | |
| AG11469 | AG11004^{ctx} | DSM 1313 Δhpt Δ0478 2366::pNA122 | — |
| | | (backbone excised) | |

Helper plasmids

| | | | |
|---|---|---|---|
| AG5119 | Top10 | F⁻mcrA Δ(mrr-hsdRMS-mcrBC) | — |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM | |
| AG8229 | Top10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA42 | |
| AG9086 | Top 10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA56G | |
| AG9087 | Top10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA58G | |
| AG9088 | Top10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA59G | |
| AG9089 | Top10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA60G | |
| AG9090 | Top10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA61G | |
| AG9091 | Top 10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA62G | |
| AG9092 | Top10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA63G | |
| AG9093 | Top10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA64G | |
| AG9094 | Top 10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA65G | |
| AG9095 | Top10 | F⁻mcrA Δ(mrr-hsdRMS-mcrBC) | Neo²⁰ |
| | ΔDCM^{E. coli} | φ80lacZΔM15 ΔlacX74 recA1 araD139 | |
| | | Δ(ara-leu)7697 galU galK λ⁻rpsL(Str^{R}) endA1 | |
| | | nupG ΔDCM/pNA66G | |

Reporter library

| | | | |
|---|---|---|---|
| AG9469 | AG8235 | DSM 1313 Δhpt Δ0478 | Tm^{15 μg/ml} |
| | | 2366::pNA28S/pNA28S | |
| AG9111 | AG8235 | DSM 1313 Δhpt Δ0478 2366::pNA29/pNA29 | Tm^{15 μg/ml} |
| AG9113 | AG8235 | DSM 1313 Δhpt Δ0478 2366::pNA30/pNA30 | Tm^{15 μg/ml} |
| AG9115 | AG8235 | DSM 1313 Δhpt Δ0478 2366::pNA31/pNA31 | Tm^{15 μg/ml} |
| AG9117 | AG8235 | DSM 1313 Δhpt Δ0478 2366::pNA32/pNA32 | Tm^{15 μg/ml} |
| AG9119 | AG8235 | DSM 1313 Δhpt Δ0478 2366::pNA33/pNA33 | Tm^{15 μg/ml} |
| AG9470 | AG8235 | DSM 1313 Δhpt Δ0478 | Tm^{15 μg/ml} |
| | | 2366::pNA34S/pNA34S | |
| AG9123 | AG8235 | DSM 1313 Δhpt Δ0478 2366::pNA35/pNA35 | Tm^{15 μg/ml} |
| AG9125 | AG8235 | DSM 1313 Δhpt Δ0478 2366::pNA36/pNA36 | Tm^{15 μg/ml} |

Homologous promoter library

| | | | |
|---|---|---|---|
| AG9469 | AG8235 | DSM 1313 Δhpt Δ0478 | Tm^{15 μg/ml} |
| | | 2366::pNA28S/pNA28S | |
| AG9104 | AG8235 | DSM 1313 Δhpt Δ0478 2366::pNA67/pNA67 | Tm^{15 μg/ml} |
| AG9471 | AG8235 | DSM 1313 Δhpt Δ0478 2366::pNA68/pNA68 | Tm^{15 μg/ml} |
| AG9169 | AG8235 | DSM 1313 Δhpt Δ0478 2366::pNA69/pNA69 | Tm^{15 μg/ml} |
| AG9170 | AG8235 | DSM 1313 Δhpt Δ0478 2366::pNA70/pNA70 | Tm^{15 μg/ml} |
| AG9171 | AG8235 | DSM 1313 Δhpt Δ0478 2366::pNA71/pNA71 | Tm^{15 μg/ml} |
| AG9172 | AG8235 | DSM 1313 Δhpt Δ0478 2366::pNA72/pNA72 | Tm^{15 μg/ml} |
| AG9173 | AG8235 | DSM 1313 Δhpt Δ0478 2366::pNA73/pNA73 | Tm^{15 μg/ml} |
| AG9174 | AG8235 | DSM 1313 Δhpt Δ0478 2366::pNA74/pNA74 | Tm^{15 μg/ml} |
| AG9467 | AG8235 | DSM 1313 Δhpt Δ0478 2366::pNA75/pNA75 | Tm^{15 μg/ml} |
| AG9175 | AG8235 | DSM 1313 Δhpt Δ0478 2366::pNA76/pNA76 | Tm^{15 μg/ml} |
| AG9176 | AG8235 | DSM 1313 Δhpt Δ0478 2366::pNA77/pNA77 | Tm^{15 μg/ml} |
| AG9177 | AG8235 | DSM 1313 Δhpt Δ0478 2366::pNA78/pNA78 | Tm^{15 μg/ml} |
| AG9178 | AG8235 | DSM 1313 Δhpt Δ0478 2366::pNA79/pNA79 | Tm^{15 μg/ml} |
| AG9462 | AG8235 | DSM 1313 Δhpt Δ0478 2366::pNA80/pNA80 | Tm^{15 μg/ml} |

Heterologous promoter library

| | | | |
|---|---|---|---|
| AG10225 | AG8235 | DSM 1313 Δhpt Δ0478 2366::pNA90/pNA90 | Tm^{15 μg/ml} |
| AG10226 | AG8235 | DSM 1313 Δhpt Δ0478 | Tm^{15 μg/ml} |
| | | 2366::pNA156/pNA156 | |
| AG10227 | AG8235 | DSM 1313 Δhpt Δ0478 | Tm^{15 μg/ml} |
| | | 2366::pNA157/pNA157 | |
| AG10228 | AG8235 | DSM 1313 Δhpt Δ0478 | Tm^{15 μg/ml} |
| | | 2366::pNA158/pNA158 | |
| AG10423 | AG8235 | DSM 1313 Δhpt Δ0478 | Tm^{15 μg/ml} |
| | | 2366::pNA159/pNA159 | |
| AG10229 | AG8235 | DSM 1313 Δhpt Δ0478 | Tm^{15 μg/ml} |
| | | 2366::pNA160/pNA160 | |
| AG10230 | AG8235 | DSM 1313 Δhpt Δ0478 | Tm^{15 μg/ml} |
| | | 2366::pNA161/pNA161 | |
| AG10231 | AG8235 | DSM 1313 Δhpt Δ0478 | Tm^{15 μg/ml} |
| | | 2366::pNA162/pNA162 | |
| AG10232 | AG8235 | DSM 1313 Δhpt Δ0478 | Tm^{15 μg/ml} |
| | | 2366::pNA163/pNA163 | |
| AG10233 | AG8235 | DSM 1313 Δhpt Δ0478 | Tm^{15 μg/ml} |
| | | 2366::pNA164/pNA164 | |
| AG10424 | AG8235 | DSM 1313 Δhpt Δ0478 | Tm^{15 μg/ml} |
| | | 2366::pNA165/pNA165 | |
| AG10425 | AG8235 | DSM 1313 Δhpt Δ0478 | Tm^{15 μg/ml} |
| | | 2366::pNA166/pNA166 | |
| AG10426 | AG8235 | DSM 1313 Δhpt Δ0478 | Tm^{15 μg/ml} |
| | | 2366::pNA167/pNA167 | |
| AG10186 | AG8235 | DSM 1313 Δhpt Δ0478 2366::pNA93/pNA93 | Tm^{15 μg/ml} |
| AG10187 | AG8235 | DSM 1313 Δhpt Δ0478 2366::pNA94/pNA94 | Tm^{15 μg/ml} |
| AG10188 | AG8235 | DSM 1313 Δhpt Δ0478 2366::pNA95/pNA95 | Tm^{15 μg/ml} |
| AG10207 | AG8235 | DSM 1313 Δhpt Δ0478 2366::pNA96/pNA96 | Tm^{15 μg/ml} |
| AG10208 | AG8235 | DSM 1313 Δhpt Δ0478 2366::pNA97/pNA97 | Tm^{15 μg/ml} |
| AG10189 | AG8235 | DSM 1313 Δhpt Δ0478 2366::pNA98/pNA98 | Tm^{15 μg/ml} |
| AG10190 | AG8235 | DSM 1313 Δhpt Δ0478 2366::pNA99/pNA99 | Tm^{15 μg/ml} |
| AG10191 | AG8235 | DSM 1313 Δhpt Δ0478 | Tm^{15 μg/ml} |
| | | 2366::pNA100/pNA100 | |
| AG10209 | AG8235 | DSM 1313 Δhpt Δ0478 | Tm^{15 μg/ml} |
| | | 2366::pNA101/pNA101 | |
| AG10192 | AG8235 | DSM 1313 Δhpt Δ0478 | Tm^{15 μg/ml} |
| | | 2366::pNA102/pNA102 | |
| AG10193 | AG8235 | DSM 1313 Δhpt Δ0478 | Tm^{15 μg/ml} |
| | | 2366::pNA103/pNA103 | |
| AG10194 | AG8235 | DSM 1313 Δhpt Δ0478 | Tm^{15 μg/ml} |
| | | 2366::pNA104/pNA104 | |
| AG9472 | AG8235 | DSM 1313 Δhpt Δ0478 2366::pNA87/pNA87 | Tm^{15 μg/ml} |
| AG9473 | AG8235 | DSM 1313 Δhpt Δ0478 2366::pNA88/pNA88 | Tm^{15 μg/ml} |
| AG9474 | AG8235 | DSM 1313 Δhpt Δ0478 2366::pNA89/pNA89 | Tm^{15 μg/ml} |
| AG9475 | AG8235 | DSM 1313 Δhpt Δ0478 2366::pNA91/pNA91 | Tm^{15 μg/ml} |
| AG11047 | AG10995 | DSM 1313 Δhpt Δ0478 | Tm^{15 μg/ml} |
| | | 2366::pNA187/pNA187 | |
| AG11350 | AG10995 | DSM 1313 Δhpt Δ0478 | Tm^{15 μg/ml} |
| | | 2366::pNA188/pNA188 | |

RBS library

| | | | |
|---|---|---|---|
| AG11343 | AG10995 | DSM 1313 Δhpt Δ0478 | Tm^{15 μg/ml} |
| | | 2366::pNA195/pNA195 | |
| AG11344 | AG10995 | DSM 1313 Δhpt Δ0478 | Tm^{15 μg/ml} |
| | | 2366::pNA196/pNA196 | |
| AG11345 | AG10995 | DSM 1313 Δhpt Δ0478 | Tm^{15 μg/ml} |
| | | 2366::pNA197/pNA197 | |
| AG11346 | AG10995 | DSM 1313 Δhpt Δ0478 | Tm^{15 μg/ml} |
| | | 2366::pNA198/pNA198 | |
| AG11294 | AG10995 | DSM 1313 Δhpt Δ0478 | Tm^{15 μg/ml} |
| | | 2366::pNA199/pNA199 | |
| AG11800 | AG10995 | DSM 1313 Δhpt Δ0478 | Tm^{15 μg/ml} |
| | | 2366::pNA280/pNA280 | |
| AG11801 | AG10995 | DSM 1313 Δhpt Δ0478 | Tm^{15 μg/ml} |
| | | 2366::pNA281/pNA281 | |
| AG11802 | AG10995 | DSM 1313 Δhpt Δ0478 | Tm^{15 μg/ml} |
| | | 2366::pNA282/pNA282 | |
| AG11803 | AG10995 | DSM 1313 Δhpt Δ0478 | Tm^{15 μg/ml} |
| | | 2366::pNA283/pNA283 | |
| AG11804 | AG10995 | DSM 1313 Δhpt Δ0478 | Tm^{15 μg/ml} |
| | | 2366::pNA284/pNA284 | |

Inducible promoter library

| | | | |
|---|---|---|---|
| AG13737 | AG8235 | DSM 1313 Δhpt Δ0478 2366::pYK-NA4/pYK- | Tm^{15 μg/ml} |
| | | NA4 | |
| AG13738 | AG8235 | DSM 1313 Δhpt Δ0478 2366::pYK- | Tm^{15 μg/ml} |
| | | NA18/pYK-NA18 | |
| AG13739 | AG8235 | DSM 1313 Δhpt Δ0478 2366::pYK4/pYK4 | Tm^{15 μg/ml} |
| AG13740 | AG8235 | DSM 1313 Δhpt Δ0478 2366::pYK17/pYK17 | Tm^{15 μg/ml} |

Riboswitch library

| | | | |
|---|---|---|---|
| AG13741 | AG8235 | DSM 1313 Δhpt Δ0478 2366::pYK-NA7/pYK- | Tm^{15 μg/ml} |
| | | NA7 | |
| AG13742 | AG8235 | DSM 1313 Δhpt Δ0478 2366::pYK-NA8/pYK- | Tm^{15 μg/ml} |
| | | NA8 | |
| AG13743 | AG8235 | DSM 1313 Δhpt Δ0478 2366::pYK-NA9/pYK- | Tm^{15 μg/ml} |
| | | NA9 | |
| AG13744 | AG8235 | DSM 1313 Δhpt Δ0478 2366::pYK- | Tm^{15 μg/ml} |
| | | NA12/pYK-NA12 | |
| AG13745 | AG8235 | DSM 1313 Δhpt Δ0478 2366::pYK- | Tm^{15 μg/ml} |
| | | NA13/pYK-NA13 | |
| AG13746 | AG8235 | DSM 1313 Δhpt Δ0478 2366::pYK- | Tm^{15 μg/ml} |
| | | NA15/pYK-NA15 | |
| AG13747 | AG8235 | DSM 1313 Δhpt Δ0478 2366::pYK- | Tm^{15 μg/ml} |
| | | NA16/pYK-NA16 | |
| AG13748 | AG8235 | DSM 1313 Δhpt Δ0478 2366::pYK- | Tm^{15 μg/ml} |
| | | NA17/pYK-NA17 | |
| AG13749 | AG8235 | DSM 1313 Δhpt Δ0478 2366::pYK5/pYK5 | Tm^{15 μg/ml} |
| AG13750 | AG8235 | DSM 1313 Δhpt Δ0478 2366::pYK7/pYK7 | Tm^{15 μg/ml} |

TABLE 2

| | | | | |
|---|---|---|---|---|
| Plasmid | | | | Selectable |
| ID | Cargo | Used for | Origin^{organism} | marker^{conc(μg/ml)} |

Homologous recombination plasmids

| | | | | |
|---|---|---|---|---|
| pNA24D | Clo1313_2366 Homologies | Replaces | p15A^{E.coli} | Tm¹⁵ |
| | flanking polyattB cassette | Clo1313_2366 | pNW33N^{ctx} | |
| | | gene with | | |
| | | polyattB cassette | | |
| | | (landing pad) | | |
| | | using | | |
| | | homologous | | |
| | | recombination | | |
| pNA153 | Clo1313_2366 Homologies | Replaces | p15A^{E.coli} | Tm¹⁵ |
| | flanking Y412MC61 attB | Clo1313_2366 | pNW33N^{ctx} | |
| | cassette | gene with | | |
| | | Y412MC61 attB | | |
| | | site using | | |
| | | homologous | | |
| | | recombination | | |

Helper plasmids

| | | | | |
|---|---|---|---|---|
| pNA42 | P^{ctx}_2366: Y412MC61 | Expresses the | p15A^{E.coli} | Neo^{20(E.coli)} |
| | | Y412MC61 | pNW33N^{ctx} | Neo^{300(ctx)} |
| | | serine | | |
| | | recombinase. Co- | | |
| | | transformed with | | |
| | | cargo containing | | |
| | | Y412MC61 attP | | |
| | | site | | |
| pNA56G | P^{ctx}_2366: BXB1 | Expresses the | p15A^{E.coli} | Neo^{20(E.coli)} |
| | | BXB1 serine | pNW33N^{ctx} | Neo^{300(ctx)} |
| | | recombinase. Co- | | |
| | | transformed with | | |
| | | cargo containing | | |
| | | BXB1 attP site. | | |
| | | Also transformed | | |
| | | into C. | | |
| | | thermocellum | | |
| | | cells with | | |
| | | genome- | | |
| | | integrated BXB1 | | |
| | | attB and BXB1 | | |
| | | attP sites for | | |
| | | backbone | | |
| | | excision | | |
| pNA58G | P^{ctx}_2366: ΦRV1 | Expresses the | p15A^{E.coli} | Neo^{20(E.coli)} |
| | | ΦRV1 serine | pNW33N^{ctx} | Neo^{300(ctx)} |
| | | recombinase. Co- | | |
| | | transformed with | | |
| | | cargo containing | | |
| | | ΦRV1 attP site | | |
| pNA59G | P^{ctx}_2366: ΦBT1 | Expresses the | p15A^{E.coli} | Neo^{20(E.coli)} |
| | | ΦBT1 serine | pNW33N^{ctx} | Neo^{300(ctx)} |
| | | recombinase. Co- | | |
| | | transformed with | | |
| | | cargo containing | | |
| | | ΦBT1 attP site | | |
| pNA60G | P^{ctx}_2366: ΦFC1 | Expresses the | p15A^{E.coli} | Neo^{20(E.coli)} |
| | | ΦFC1 serine | pNW33N^{ctx} | Neo^{300(ctx)} |
| | | recombinase. Co- | | |
| | | transformed with | | |
| | | cargo containing | | |
| | | ΦFC1 attP site | | |
| pNA64G | P^{ctx}_2366: ΦA118 | Expresses the | p15A^{E.coli} | Neo^{20(E.coli)} |
| | | ΦA118 serine | | |
| | | recombinase. Co- | | |
| | | transformed with | | |
| | | cargo containing | | |
| | | ΦA118 attP site | | |
| pNA62G | P^{ctx}_2366: ΦMR11 | Expresses the | p15A^{E.coli} | Neo^{20(E.coli)} |
| | | ΦMR11 serine | pNW33N^{ctx} | Neo^{300(ctx)} |
| | | recombinase. Co- | | |
| | | transformed with | | |
| | | cargo containing | | |
| | | ΦMR11 attP site | | |
| pNA63G | P^{ctx}_2366: Φ370 | Expresses the | p15A^{E.coli} | Neo^{20(E.coli)} |
| | | Φ370 serine | pNW33N^{ctx} | Neo^{300(ctx)} |
| | | recombinase. Co- | | |
| | | transformed with | | |
| | | cargo containing | | |
| | | Φ370 attP site | | |
| pNA64G | P^{ctx}_2366: TG1 | Expresses the | p15A^{E.coli} | Neo^{20(E.coli)} |
| | | TG1 serine | pNW33N^{Ctx} | Neo^{300(ctx)} |
| | | recombinase. Co- | | |
| | | transformed with | | |
| | | cargo containing | | |
| | | TG1 attP site | | |
| pNA65G | P^{ctx}_2366: R4 | Expresses the R4 | p15A^{E.coli} | Neo^{20(E.coli)} |
| | | serine | pNW33N^{ctx} | Neo^{300(ctx)} |
| | | recombinase. Co- | | |
| | | transformed with | | |
| | | cargo containing | | |
| | | R4 attP site | | |
| pNA66G | P^{ctx}_2366: BL3 | Expresses the | p15A^{E.coli} | Neo^{20(E.coli)} |
| | | BL3 serine | pNW33N^{ctx} | Neo^{300(ctx)} |
| | | recombinase. Co- | | |
| | | transformed with | | |
| | | cargo containing | | |
| | | BL3 attP site | | |

Cargo plasmids

| | | | | |
|---|---|---|---|---|
| pHS96 | Poly attP cassette | Cargo plasmid | p15AE.coli | Tm15 |
| | | for testing SAGE | | |
| | | and t-SAGE | | |
| pNA28S | P^{ctx}_1194: sf-GFP | Reporter library | ColE1^{E.coli} | Tm¹⁵ |
| pNA29 | P^{ctx}_1194: cagFbFp-Version1 | Reporter library | ColE1^{E.coli} | Tm¹⁵ |
| pNA30 | P^{ctx}_1194: cagFbFp-Version2 | Reporter library | ColE1^{E.coli} | Tm¹⁵ |
| pNA31 | p^{ctx}_1194: mrFbFp-Version1 | Reporter library | ColE1^{E.coli} | Tm¹⁵ |
| pNA32 | P^{ctx}_1194: YNP3FbFp- | Reporter library | ColE1^{E.coli} | Tm¹⁵ |
| | Version1 | | | |
| pNA33 | P^{ctx}_1194: codon optimized | Reporter library | ColE1^{E.coli} | Tm¹⁵ |
| | superfolder GFP | | | |
| pNA34S | P^{ctx}_1194: YNP3FbFp- | Reporter library | ColE1^{E.coli} | Tm¹⁵ |
| | Version2 | | | |
| pNA35 | P^{ctx}_1194: mKATE | Reporter library | ColE1^{E.coli} | Tm¹⁵ |
| pNA36 | p^{ctx}_1194: codon optimized | Reporter library | ColE1^{E.coli} | Tm¹⁵ |
| | mKATE | | | |
| pNA67 | P^{ctx}_1954(1): sf-GFP | Homologous | ColE1^{E.coli} | Tm¹⁵ |
| | | promoter library | | |
| pNA68 | P^{ctx}_1954(2): sf-GFP | Homologous | ColE1^{E.coli} | Tm¹⁵ |
| | | promoter library | | |
| pNA69 | p^{ctx}_2090(eno): sf-GFP | Homologous | ColE1^{E.coli} | Tm¹⁵ |
| | | promoter library | | |
| pNA70 | P^{ctx}_2095(gapDH1): sf-GFP | Homologous | ColE1^{E.coli} | Tm¹⁵ |
| | | promoter library | | |
| pNA71 | P^{ctx}_2095(gapDH2): sf-GFP | Homologous | ColE1^{E.coli} | Tm¹⁵ |
| | | promoter library | | |
| pNA72 | P^{ctx}_0307: sf-GFP | Homologous | ColE1^{E.coli} | Tm¹⁵ |
| | | promoter library | | |
| pNA73 | P^{ctx}_0544: sf-GFP | Homologous | ColE1^{E.coli} | Tm¹⁵ |
| | | promoter library | | |
| pNA74 | P^{ctx}_0815: sf-GFP | Homologous | ColE1^{E.coli} | Tm¹⁵ |
| | | promoter library | | |
| pNA75 | P^{ctx}_0966: sf-GFP | Homologous | ColE1^{E.coli} | Tm¹⁵ |
| | | promoter library | | |
| pNA76 | P^{ctx}_2463: sf-GFP | Homologous | ColE1^{E.coli} | Tm¹⁵ |
| | | promoter library | | |
| pNA77 | p^{ctx}_2638: sf-GFP | Homologous | ColE1^{E.coli} | Tm¹⁵ |
| | | promoter library | | |
| pNA78 | P^{ctx}_2926: sf-GFP | Homologous | ColE1^{E.coli} | Tm¹⁵ |
| | | promoter library | | |
| pNA79 | P^{ctx}_3011(1): sf-GFP | Homologous | ColE1^{E.coli} | Tm¹⁵ |
| | | promoter library | | |
| pNA80 | P^{ctx}_3011(2): sf-GFP | Homologous | ColE1^{E.coli} | Tm¹⁵ |
| | | promoter library | | |
| pNA87 | P^{gth}_Rp1S: sf-GFP | Heterologous | ColE1^{E.coli} | Tm¹⁵ |
| | | promoter library | | |
| pNA88 | P^{gth}_Ldh: sf-GFP | Heterologous | ColE1^{E.coli} | Tm¹⁵ |
| | | promoter library | | |
| pNA89 | P^{tsh}_0046(PFOR): sf-GFP | Heterologous | ColE1^{E.coli} | Tm¹⁵ |
| | | promoter library | | |
| pNA90 | p^{spv}_1266(CAT): sf-GFP | Heterologous | ColE1^{E.coli} | Tm¹⁵ |
| | | promoter library | | |
| pNA91 | P^{ate}_2105: sf-GFP | Heterologous | ColE1^{E.coli} | Tm¹⁵ |
| | | promoter library | | |
| pNA93 | p^{gtn}_2651: sf-GFP | Heterologous | ColE1^{E.coli} | Tm¹⁵ |
| | | promoter library | | |
| pNA94 | p^{gtn}_3007: sf-GFP | Heterologous | ColE1^{E.coli} | Tm¹⁵ |
| | | promoter library | | |
| pNA95 | p^{gtn}_1794: sf-GFP | Heterologous | ColE1^{E.coli} | Tm¹⁵ |
| | | promoter library | | |
| pNA96 | p^{gtn}_0611: sf-GFP | Heterologous | ColE1^{E.coli} | Tm¹⁵ |
| | | promoter library | | |
| pNA97 | p^{gtn}_2022: sf-GFP | Heterologous | ColE1^{E.coli} | Tm¹⁵ |
| | | promoter library | | |
| pNA98 | p^{gtn}_3244: sf GFP | Heterologous | ColE1^{E.coli} | Tm¹⁵ |
| | | promoter library | | |
| pNA99 | p^{gtn}_0824: sf-GFP | Heterologous | ColE1^{E.coli} | Tm¹⁵ |
| | | promoter library | | |
| pNA100 | p^{gtn}_1780: sf-GFP | Heterologous | ColE1^{E.coli} | Tm¹⁵ |
| | | promoter library | | |
| pNA101 | p^{gtn}_2506: sf-GFP | Heterologous | ColE1^{E.coli} | Tm¹⁵ |
| | | promoter library | | |
| pNA102 | p^{gtn}_0914: sf-GFP | Heterologous | ColE1^{E.coli} | Tm¹⁵ |
| | | promoter library | | |
| pNA103 | p^{gtn}_0489: sf-GFP | Heterologous | ColE1^{E.coli} | Tm¹⁵ |
| | | promoter library | | |
| pNA104 | p^{gtn}_2690: sf-GFP | Heterologous | ColE1^{E.coli} | Tm¹⁵ |
| | | promoter library | | |
| pNA122 | P^{ctx}_1194: sf-GFP | Testing Backbone | ColE1^{E.coli} | Tm¹⁵ |
| | | excision using | | |
| | | BXB1 integrase | | |
| pNA156 | p^{ccl}_2510: sf-GFP | Heterologous | ColE1^{E.coli} | Tm¹⁵ |
| | | promoter library | | |
| pNA157 | p^{ccl}_0388: sf-GFP | Heterologous | ColE1^{E.coli} | Tm¹⁵ |
| | | promoter library | | |
| pNA158 | p^{ccl}_0388: sf-GFP | Heterologous | ColE1^{E.coli} | Tm¹⁵ |
| | | promoter library | | |
| pNA159 | p^{ccl}_2515: sf-GFP | Heterologous | ColE1^{E.coli} | Tm¹⁵ |
| | | promoter library | | |
| pNA160 | p^{ccl}_0877: sf-GFP | Heterologous | ColE1^{E.coli} | Tm¹⁵ |
| | | promoter library | | |
| pNA161 | p^{ccl}_0877: sf-GFP | Heterologous | ColE1^{E.coli} | Tm¹⁵ |
| | | promoter library | | |
| pNA162 | p^{ccl}_0867: sf-GFP | Heterologous | ColE1^{E.coli} | Tm¹⁵ |
| | | promoter library | | |
| pNA163 | p^{ccl}_1058: sf-GFP | Heterologous | ColE1^{E.coli} | Tm¹⁵ |
| | | promoter library | | |
| pNA164 | P^{ccl}_4202: sf-GFP | Heterologous | ColE1^{E.coli} | Tm¹⁵ |
| | | promoter library | | |
| pNA165 | p^{ccl}_4203: sf-GFP | Heterologous | ColE1^{E.coli} | Tm¹⁵ |
| | | promoter library | | |
| pNA166 | p^{ccl}_0018: sf-GFP | Heterologous | ColE1^{E.coli} | Tm¹⁵ |
| | | promoter library | | |
| pNA167 | p^{ccl}_0416: sf-GFP | Heterologous | ColE1^{E.coli} | Tm¹⁵ |
| | | promoter library | | |
| pNA187 | Pₜₛₕ_0068: sf-GFP | Heterologous | ColE1^{E.coli} | Tm¹⁵ |
| | | promoter library | | |
| pNA188 | Pₜₛₕ_0530: sf-GFP | Heterologous | ColE1^{E.coli} | Tm¹⁵ |
| | | promoter library | | |
| pNA195 | p1194agggggaaaaaaacATG | RBS library | ColE1^{E.coli} | Tm¹⁵ |
| | (SEQ ID NO: 27) | (Decreasing | | |
| | | distance) | | |
| pNA196 | p1194agggggaaaaaaaATG | RBS library | ColE1^{E.coli} | Tm¹⁵ |
| | (SEQ ID NO: 28) | (Decreasing | | |
| | | distance) | | |
| pNA197 | p1194agggggaaaaaaATG | RBS library | ColE1^{E.coli} | Tm¹⁵ |
| | (SEQ ID NO: 29) | (Decreasing | | |
| | | distance) | | |
| pNA198 | p1194agggggaaaaaATG | RBS library | ColE1^{E.coli} | Tm¹⁵ |
| | (SEQ ID NO: 30) | (Decreasing | | |
| | | distance) | | |
| pNA199 | p1194agggggaaaaATG | RBS library | ColE1^{E.coli} | Tm¹⁵ |
| | (SEQ ID NO: 31) | (Decreasing | | |
| | | distance) | | |
| pNA280 | p1194agggggaaaaaaactcATG | RBS library | ColE1^{E.coli} | Tm¹⁵ |
| | (SEQ ID NO: 32) | (Increasing | | |
| | | distance) | | |
| pNA281 | p1194agggggaaaaaaactctATG | RBS library | ColE1^{E.coli} | Tm¹⁵ |
| | (SEQ ID NO: 33) | (Increasing | | |
| | | distance) | | |
| pNA282 | p1194agggggaaaaaaactctcATG | RBS library | ColE1^{E.coli} | Tm¹⁵ |
| | (SEQ ID NO: 34) | (Increasing | | |
| | | distance) | | |
| pNA283 | p1194agggggaaaaaaactctctATG | RBS library | ColE1^{E.coli} | Tm¹⁵ |
| | (SEQ ID NO: 35) | (Increasing | | |
| | | distance) | | |
| pNA284 | p1994aggaggaaaaaaactATG | RBS library | ColE1^{E.coli} | Tm¹⁵ |
| | (SEQ ID NO: 36) | (canonical) | | |
| pYK- | p^{cdl}_2899: sf-GFP; P^{ctx}_1194: | Inducible | ColE1^{E.coli} | Tm¹⁵ |
| NA4 | CDR20291_2900 (xlyR) | promoter library | | |
| pYK- | P^{ate}_0603: sf-GFP; P^{ctx}_1194: | Inducible | ColE1^{E.coli} | Tm¹⁵ |
| NA18 | Athe_0617 (xylR) | promoter library | | |
| pYK4 | p^{ppf}_2899: sf-GFP; P^{ctx}_1194: | Inducible | ColE1^{E.coli} | Tm¹⁵ |
| | Pput_2908 (cymR) | promoter library | | |
| pYK17 | p^{eco}_0063: sf-GFP; P^{eco}_0064: | Inducible | ColE1^{E.coli} | Tm¹⁵ |
| | b0064 (araC) | promoter library | | |
| pYK- | P^{ctx}_1194: pbuE(WT): sf-GFP | Riboswitch | ColE1^{E.coli} | Tm¹⁵ |
| NA7 | | library | | |
| pYK- | P^{ctx}_1194: pbuE(P1 = 8): | Riboswitch | ColE1^{E.coli} | Tm¹⁵ |
| NA8 | sf-GFP | library | | |
| pYK- | P^{ctx}_1194: pbuE(P1 = 10): | Riboswitch | ColE1^{E.coli} | Tm¹⁵ |
| NA9 | sf-GFP | library | | |
| pYK- | p^{ctx}_2095(gapDH1): | Riboswitch | ColE1^{E.coli} | Tm¹⁵ |
| NA12 | pbuE(WT): sf-GFP | library | | |
| pYK- | P^{ctx}_2095(gapDH1): | Riboswitch | ColE1^{E.coli} | Tm¹⁵ |
| NA13 | pbuE(P1 = 8): sf-GFP | library | | |
| pYK- | P^{tsh}_0068: pbuE(WT): sf-GFP | Riboswitch | ColE1^{E.coli} | Tm¹⁵ |
| NA15 | | library | | |
| pYK- | P^{tsh}_0068: pbuE(P1 = 8): | Riboswitch | ColE1^{E.coli} | Tm¹⁵ |
| NA16 | sf-GFP | library | | |
| pYK- | P^{tsh}_0068: pbuE(P1 = 10): | Riboswitch | ColE1^{E.coli} | Tm¹⁵ |
| NA17 | sf-GFP | library | | |
| pYK5 | P^{ctx}_1194: F-(bce): sf-GFP | Riboswitch | ColE1^{E.coli} | Tm¹⁵ |
| | | library | | |
| pYK7 | P^{ctx}_1194: F-(tpt): sf-GFP | Riboswitch | ColE1^{E.coli} | Tm¹⁵ |
| | | library | | |

1. A genetically engineered thermophile bacterial cell comprising at least one att site in its chromosome wherein the att site is one member of a pair of attB and attP recombination sites.

2. The thermophile bacterial cell of claim 1, wherein;
i) the cell expresses a thermophilic site-specific recombinase that recognizes the pair of attB and attP recombination sites; and/or
ii) the at least one att site comprises multiple att sites, each being a member of a pair of attB and attP recombination sites recognized by different site-specific recombinases.

3. (canceled)

4. A system for stable insertion of a heterologous DNA, the system comprising:
a thermophile bacterial cell comprising at least one att site in its chromosome wherein the att site is one member of a pair of attB and attP recombination sites; and
a cargo plasmid comprising the heterologous DNA and a corresponding att site, wherein the corresponding att site is the other member of the pair of attB and attP recombination sites;
wherein the cargo plasmid is capable of insertion into the chromosome of the thermophile at the at least one att site via recombination with the corresponding att site on the cargo plasmid; and
wherein the recombination is mediated by a thermophilic site-specific recombinase that recognizes the pair of attB and attP recombination sites, and results in the formation of an attL site and an attR site, wherein the attL and attR sites are not substrates for further recombination.

5. The system of claim 4, wherein the thermophile bacterial cell comprises a native att site in its chromosome.

6. The system of claim 4, wherein the thermophile bacterial cell comprises a genetically engineered att site in its chromosome.

7. The system of claim 4, wherein the thermophile bacterial cell comprises both native att and genetically engineered att cites.

8. The system of claim 4, wherein the thermophile bacterial cell expresses the thermophilic site-specific recombinase.

9. The system of claim 4, further comprising a nucleic acid encoding the thermophilic site-specific recombinase.

10. The system of claim 9, wherein the nucleic acid encoding the thermophilic site-specific recombinase is provided on a helper plasmid.

11. The system of claim 4, wherein the cargo plasmid further comprises a selectable marker gene.

12. The system of claim 11, wherein the selectable marker gene is flanked by another pair of pair of attB and attP recombination sites recognized by another thermophilic site-specific recombinase.

13. The system of claim 4, wherein the thermophilic site-specific recombinase and the another thermophilic site-specific recombinase are a serine recombinase or a tyrosine recombinase.

14. The system of claim 13, wherein the serine recombinase is selected from Y412MC61, BXB1, and TG1, and homologs thereof.

15.-16. (canceled)

17. The system of claim 4, wherein;
(i) the thermophile bacterium cell is a cell selected from the genus of Bacillus, Geobacillus, Paenebacillus, Clostridium, Anaerocellum, Caldicellulosiruptor, Thermus, Pyrococcus, Thermococcus, Thermoanaerobacter, Thermoanaerobacterium, Herbinix, Acetivibrio, or Acidothermus; or
(ii) the thermophile bacterium cell is a Clostridium thermocellum, Geobacillus thermoglucosidasius, or Bacillus licheniformis.

18. (canceled)

19. The system of claim 4, wherein the thermophile bacterial cell comprises multiple att sites in its chromosome, and the system comprises another cargo plasmid comprising another corresponding att site.

20. A method for thermostable insertion of a heterologous DNA into a chromosome of an organism, the method comprising:
a) transfecting a cargo plasmid into a thermophile bacterial cell comprising at least one att site in its chromosome wherein the att site is one member of a pair of attB and attP recombination sites,
wherein the thermophile bacterial cell expresses a thermophilic site-specific recombinase that recognizes the pair of attB and attP recombination sites;
wherein the cargo plasmid comprising a corresponding att recombination site, wherein the corresponding att recombination site is the other member of the pair of attB and attP recombination sites;
wherein the cargo plasmid comprises the heterologous DNA;
wherein the cargo plasmid is capable of insertion into the chromosome of the thermophile bacterial cell at the at least one att site via recombination with the at least one corresponding att site on the cargo plasmid; and
wherein the recombination results in the formation of an attL site and an attR site, wherein the attL and attR sites are not substrates for further recombination,
wherein the insertion is a site-specific recombination at a temperature at or above 50° C. and is thermostable; and
b) selecting a thermophile bacterial cell wherein the heterologous DNA is inserted into the chromosome of the thermophile bacterial cell.

21.-32. (canceled)

33. A thermophile bacterial cell made through the method of claim 20.

34.-45. (canceled)


SEQUENCE LISTING (36 sequences)

SEQ ID NO: 1 - DNA, 1389 bp, synthetic construct
  misc_feature 1..1389: note = Y412MC61 recombinase
  source 1..1389: mol_type = other DNA
atgaaagtggccatttatgttcgtgttagcaccgatgaacaggccaaagaaggttttagcattccggcacagcgtgaacgtctgcgtgcattttgtgcaagccagggttgggaaattgtgcaagaatatattgaagaaggttggagcgcaaaagatctggatcgtccgcagatgcagcgtctgctgaaagatatcaaaaaaggcaacattgatattgtgctggtgtatcgtctggatcgcctgacccgtagcgttctggatctgtatctgctgctgcagacctttgaaaaatacaatgtggcatttcgtagcgccaccgaagtttatgataccagcaccgcaatgggtcgtctgtttattaccctggttgcagcactggcacagtgggaacgtgaaaatctggcagaacgtgttaaatttggtatcgagcagatgatcgatgaaggtaaaaaaccgggtggtcatagcccgtatggttacaaatttgataaagacttcaattgcaccattattgaggaagaagcagacgttgttcgtatgatctatcgcatgtattgtgatggttatggctatcgtagcattgcagatcgtctgaatgaactgatggttaaaccgcgtattgccaaagaatggaatcataatagcgtgcgtgatatcctgaccaacgatatctatattggcacctatcgttggggtgataaagttgttccgaataatcatccgcctattattagcgaaaccctgttcaaaaaagcccagaaagaaaaagaaaaacgtggcgttgatcgtaaacgcgttggtaaatttctgtttaccggtctgctgcagtgtggtaattgtggtggccataaaatgcagggccattttgataaacgtgagcagaaaacctattaccgttgtaccaaatgtcaccgcattaccaacgaaaaaaacattctggaaccgctgctggatgaaattcagctgctgattaccagcaaagaatactttatgagcaaattcagcgaccgctatgatcagcaagaggttgttgatgttagcgcactgacaaaagaactggaaaaaatcaaacgccagaaagagaaatggtacgatctgtatatggatgatcgtaacccgattccgaaagaagaactgtttgccaaaattaacgaactgaacaaaaaagaagaagaaatctatagcaagctgagcgaagtggaagaagataaagaaccggttgaagagaaatataaccgcctgagcaaaatgatcgattttaaacagcagtttgagcaggccaacgactttaccaaaaaagagctgctgttcagcatcttcgaaaagattgtgatttatcgcgagaaaggcaagctgaaaaaaatcaccctggattacaccctgaaataa

SEQ ID NO: 2 - protein, 462 aa, synthetic construct
  REGION 1..462: note = Y412MC61 recombinase
MKVAIYVRVSTDEQAKEGFSIPAQRERLRAFCASQGWEIVQEYIEEGWSAKDLDRPQMQRLLKDIKKGNIDIVLVYRLDRLTRSVLDLYLLLQTFEKYNVAFRSATEVYDTSTAMGRLFITLVAALAQWERENLAERVKFGIEQMIDEGKKPGGHSPYGYKFDKDFNCTIIEEEADVVRMIYRMYCDGYGYRSIADRLNELMVKPRIAKEWNHNSVRDILTNDIYIGTYRWGDKVVPNNHPPIISETLFKKAQKEKEKRGVDRKRVGKFLFTGLLQCGNCGGHKMQGHFDKREQKTYYRCTKCHRITNEKNILEPLLDEIQLLITSKEYFMSKFSDRYDQQEVVDVSALTKELEKIKRQKEKWYDLYMDDRNPIPKEELFAKINELNKKEEEIYSKLSEVEEDKEPVEEKYNRLSKMIDFKQQFEQANDFTKKELLFSIFEKIVIYREKGKLKKITLDYTLK

SEQ ID NO: 3 - DNA, 1503 bp, synthetic construct
  misc_feature 1..1503: note = BXB1 Integrase
  source 1..1503: mol_type = other DNA
atgagagctttggttgtgatcagacttagtagagtaacagatgcgactacttctcctgaaaggcagttggaaagttgtcaacaactctgcgctcaaagaggatgggatgtcgttggagttgcagaggatttagatgtatccggcgctgttgaccctttcgatagaaaaagaagacctaatttagcaagatggctagcatttgaagagcagccttttgatgttatagttgcctatagagttgacagattaacaagatctataagacaccttcaacaattagtccattgggcagaggatcataaaaaacttgtggtttcggctacagaagcacattttgatacaacaaccccatttgctgcagtagttattgccttaatgggaacagttgctcaaatggaacttgaagctattaaggaaagaaatagaagtgctgcccactttaatataagagcgggaaagtacagaggatcactccctccttggggatatctacctactagggtagatggagaatggaggctcgtgccagatccagttcaaagagaaagaatattagaagtatatcatagagtagtagacaatcatgagcctcttcatcttgtagctcatgatctaaaccgacgaggcgtgttatcacctaaggattattttgcccagctacagggcagggagcctcaaggaagagagtggagtgctacagctttaaagaggtctatgataagcgaagctatgcttgggtatgctaccttaaatggaaaaaccgtaagagatgatgatggtgctccattagttagagctgaaccaattcttactagagaacaattggaagcacttagggcagagcttgtaaaaactagccgagcaaaacctgctgtttctactccatctctgctgcttagagttttattttgtgctgtttgcggtgagccagcatataaatttgcaggtggaggaagaaagcacccaagatatagatgtagaagcatgggttttccaaaacattgtggaaatggtacagtagcaatggcagagtgggatgcattctgtgaagaacaagttttggatttattaggagatgctgaacgtcttgaaaaagtatgggttgcgggtagtgattctgcagtagaattagctgaagtaaacgctgaattagtagatttaactagtttaataggttcacccgcgtaccgggcaggatcaccacagagagaagctctcgatgcaaggattgcagcattagcagctagacaagaagagcttgaaggtttagaggcaagaccgtcaggttgggaatggagagaaacggggcaaagatttggtgattggtggagagaacaagatacggcagctaaaaatacttggttaagatcaatgaatgttagattaacttttgatgtacgcggcggattgacaagaacaatagattttggagaccttcaggaatatgaacaacatcttcgtcttggttcagtagttgaaagactacatacaggaatgagttaa

SEQ ID NO: 4 - protein, 500 aa, synthetic construct [sequence omitted]

SEQ ID NO: 5 - DNA, 1860 bp, synthetic construct
  misc_feature 1..1860: note = TG1 Integrase
  source 1..1860: mol_type = other DNA
atggttattttagctggtggatatgatagacagtcggcagaacgtgaaaattcaagtacagcttctccagcaacacaaagagctgcaaatagaggtaaggcagaagctctagcaaaagaatatgcaagggatggtgtagaagttaaatggcttggacattttagtgaggcacctggtacaagcgcctttacaggagttgatagacctgaatttaatcgtatacttgatatgtgtaggaacagagaaatgaatatgataattgttcattatatttctaggttgagtagggaagaacctcttgatattattccagtagttacggagcttcttcgattgggagtgacaatagtatcagttaatgaaggtacctttagaccaggtgaaatgatggatttgatacacttaataatgaggttacaagcctcacacgatgaaagtaaaaataaatctgtagctgtgtctaatgctaaggaacttgcaaagagattgggaggacatacaggctcaacaccttacggatttgatactgttgaagagatggtaccaaatccagaagatggtggaaaattagttgctattagaaggcttgtacccagcgctcatacttgggaaggagctcatggatctgagggagcagtaataagatgggcttggcaagaaataaaaacacatagagatacaccgtttaaaggggggggtgctggtagctttcatcctggttctttaaatggattatgtgaaagactttatagagataaggtaccaacaagaggaactctagtgggaaaaaaacgcgccggttccgattgggatccaggagtactaaaaagagtgttatcagaccctagaattgcaggctatcaggccgatatagcttataaagtaagagcagatggaagtcgaggtggattttctcattacaaaataagaagggaccctgtaactatggagcctctgactcttcccggctttgaaccatatataccacctgcagagtggtgggaattacaggagtggcttcaaggaaggggtagaggcaagggacaatatagaggacagagcctcttatcagcaatggatgttttatattgctatggatcaggacagttggatccagaaactggttactccaacggttcgactatggcaggaaatgttagagaaggggatcaagcacataaaagtagttatgcatgtaaatgtccaagaagagttcatgatgggagttcttgctcaataacaatgcataatttagatccttatattgttggagcaatatttgcaagaattaccgctttcgatccagctgaccctgatgatttagaaggagacactgcagcgttaatgtatgaagctgctagaaggtggggtgctactcatgaaagaccagaactcaaaggtcaacgttctgaacttatggctcaaagagcagatgctgtaaaagcacttgaagaattatatgaagataaaagaaatggaggttacagaagtgctatgggaagacgggcttttctagaggaggaagcagcgctaactttaagaatggaaggggccgaggaaagactgagacaattagatgctgctgatagtccagtcttaccaattggagaatggttaggtgaccgtggatcagatccaactggacctggctcatggtgggcacttgctccgctggaggatcgacgtgcatttgttagattatttgtagatagaatagaagtaataaagcttcctaaaggtgttcaaagacctgggagagttcctcctatagctgatcgggttagaatccactgggcaaagcctaaggtggaagaggaaacagaaccagagacacttaacggtttcacggcagcagcataa

SEQ ID NO: 6 - protein, 619 aa, synthetic construct
  REGION 1..619: note = TG1 Integrase
MVILAGGYDRQSAERENSSTASPATQRAANRGKAEALAKEYARDGVEVKWLGHFSEAPGTSAFTGVDRPEFNRILDMCRNREMNMIIVHYISRLSREEPLDIIPVVTELLRLGVTIVSVNEGTFRPGEMMDLIHLIMRLQASHDESKNKSVAVSNAKELAKRLGGHTGSTPYGFDTVEEMVPNPEDGGKLVAIRRLVPSAHTWEGAHGSEGAVIRWAWQEIKTHRDTPFKGGGAGSFHPGSLNGLCERLYRDKVPTRGTLVGKKRAGSDWDPGVLKRVLSDPRIAGYQADIAYKVRADGSRGGFSHYKIRRDPVTMEPLTLPGFEPYIPPAEWWELQEWLQGRGRGKGQYRGQSLLSAMDVLYCYGSGQLDPETGYSNGSTMAGNVREGDQAHKSSYACKCPRRVHDGSSCSITMHNLDPYIVGAIFARITAFDPADPDDLEGDTAALMYEAARRWGATHERPELKGQRSELMAQRADAVKALEELYEDKRNGGYRSAMGRRAFLEEEAALTLRMEGAEERLRQLDAADSPVLPIGEWLGDRGSDPTGPGSWWALAPLEDRRAFVRLFVDRIEVIKLPKGVQRPGRVPPIADRVRIHWAKPKVEEETEPETLNGFTAAA

SEQ ID NO: 7 - DNA, 62 bp, synthetic construct
  misc_feature 1..62: note = Y412MC61_attB
  source 1..62: mol_type = other DNA
agacgagaaacgttccgtccgtctgggtcagttgggcaaagttgatgaccgggtcgtccgtt

SEQ ID NO: 8 - DNA, 66 bp, synthetic construct
  misc_feature 1..66: note = Y412MC61_attP
  source 1..66: mol_type = other DNA
gtgttataaacctgtgtgagagttaagtttacatgcctaaccttaacttttacgcaggttcagctt

SEQ ID NO: 9 - DNA, 50 bp, synthetic construct
  misc_feature 1..50: note = BXB1_attB
  source 1..50: mol_type = other DNA
tcggccggcttgtcgacgacggcggtctccgtcgtcaggatcatccgggc

SEQ ID NO: 10 - DNA, 58 bp, synthetic construct
  misc_feature 1..58: note = BXB1_attP
  source 1..58: mol_type = other DNA
gtcgtggtttgtctggtcaaccaccgcggtctcagtggtgtacggtacaaaccccgac

SEQ ID NO: 11 - DNA, 46 bp, synthetic construct
  misc_feature 1..46: note = TG1_attB
  source 1..46: mol_type = other DNA
gatcagctccgcgggcaagaccttctccttcacggggtggaaggtc

SEQ ID NO: 12 - DNA, 67 bp, synthetic construct
  misc_feature 1..67: note = TG1_attP
  source 1..67: mol_type = other DNA
tcaaccccgttccagcccaacagtgttagtctttgctcttacccagttgggcgggatagcctgcccg

SEQ ID NO: 13 - DNA, 65 bp, synthetic construct
  misc_feature 1..65: note = Y412MC61_attL
  source 1..65: mol_type = other DNA
agacgagaaacgttccgtccgtctgggtcagttgcctaaccttaacttttacgcaggttcagctt

SEQ ID NO: 14 - DNA, 63 bp, synthetic construct
  misc_feature 1..63: note = Y412MC61_attR
  source 1..63: mol_type = other DNA
gtgttataaacctgtgtgagagttaagtttacatgggcaaagttgatgaccgggtcgtccgtt

SEQ ID NO: 15 - DNA, 54 bp, synthetic construct
  misc_feature 1..54: note = BXB1_attL
  source 1..54: mol_type = other DNA
tcggccggcttgtcgacgacggcggtctcagtggtgtacggtacaaaccccgac

SEQ ID NO: 16 - DNA, 54 bp, synthetic construct
  misc_feature 1..54: note = BXB1_attR
  source 1..54: mol_type = other DNA
gtcgtggtttgtctggtcaaccaccgcggtctccgtcgtcaggatcatccgggc

SEQ ID NO: 17 - DNA, 58 bp, synthetic construct
  misc_feature 1..58: note = TG1_attL
  source 1..58: mol_type = other DNA
gatcagctccgcgggcaagacctttgctcttacccagttgggcgggatagcctgcccg

SEQ ID NO: 18 - DNA, 55 bp, synthetic construct
  misc_feature 1..55: note = TG1_attR
  source 1..55: mol_type = other DNA
tcaaccccgttccagcccaacagtgttagtcttctccttcacggggtggaaggtc

SEQ ID NO: 19 - DNA, 18 bp, synthetic construct
  misc_feature 1..18: note = PCR Primer 10
  source 1..18: mol_type = other DNA
cggtttctttaccagact

SEQ ID NO: 20 - DNA, 20 bp, synthetic construct
  misc_feature 1..20: note = PCR Primer 11
  source 1..20: mol_type = other DNA
ggtattttacccttgtcgtt

SEQ ID NO: 21 - DNA, 22 bp, synthetic construct
  misc_feature 1..22: note = PCR Primer 13
  source 1..22: mol_type = other DNA
ctctgctactcgtttttcaatc

SEQ ID NO: 22 - DNA, 20 bp, synthetic construct
  misc_feature 1..20: note = PCR Primer 14
  source 1..20: mol_type = other DNA
gggaaggatttgaggttagg

SEQ ID NO: 23 - DNA, 23 bp, synthetic construct
  misc_feature 1..23: note = PCR Primer 49
  source 1..23: mol_type = other DNA
ggcaccaataactgccttaaaga

SEQ ID NO: 24 - DNA, 23 bp, synthetic construct
  misc_feature 1..23: note = PCR Primer 50
  source 1..23: mol_type = other DNA
cgatgataaacaaaggacggttc

SEQ ID NO: 25 - DNA, 15 bp, synthetic construct
  misc_feature 1..15: note = RBS Spacer Sequence
  source 1..15: mol_type = other DNA
agggggaaaaaaact

SEQ ID NO: 26 - DNA, 15 bp, synthetic construct
  misc_feature 1..15: note = Consensus Sequence RBS Spacer Sequence
  source 1..15: mol_type = other DNA
aggaggaaaaaaact

SEQ ID NO: 27 - DNA, 17 bp, synthetic construct
  misc_feature 1..17: note = pNA195 plasmid
  source 1..17: mol_type = other DNA
agggggaaaaaaacatg

SEQ ID NO: 28 - DNA, 16 bp, synthetic construct
  misc_feature 1..16: note = pNA196 plasmid
  source 1..16: mol_type = other DNA
agggggaaaaaaaatg

SEQ ID NO: 29 - DNA, 15 bp, synthetic construct
  misc_feature 1..15: note = pNA197 plasmid
  source 1..15: mol_type = other DNA
agggggaaaaaaatg

SEQ ID NO: 30 - DNA, 14 bp, synthetic construct
  misc_feature 1..14: note = pNA198 plasmid
  source 1..14: mol_type = other DNA
agggggaaaaaatg

SEQ ID NO: 31 - DNA, 13 bp, synthetic construct
  misc_feature 1..13: note = pNA199 plasmid
  source 1..13: mol_type = other DNA
agggggaaaaatg

SEQ ID NO: 32 - DNA, 19 bp, synthetic construct
  misc_feature 1..19: note = pNA280 plasmid
  source 1..19: mol_type = other DNA
agggggaaaaaaactcatg

SEQ ID NO: 33 - DNA, 20 bp, synthetic construct
  misc_feature 1..20: note = pNA281 plasmid
  source 1..20: mol_type = other DNA
agggggaaaaaaactctatg

SEQ ID NO: 34 - DNA, 21 bp, synthetic construct
  misc_feature 1..21: note = pNA282 plasmid
  source 1..21: mol_type = other DNA
agggggaaaaaaactctcatg

SEQ ID NO: 35 - DNA, 22 bp, synthetic construct
  misc_feature 1..22: note = pNA283 plasmid
  source 1..22: mol_type = other DNA
agggggaaaaaaactctctatg

SEQ ID NO: 36 - DNA, 18 bp, synthetic construct
  misc_feature 1..18: note = pNA284 plasmid
  source 1..18: mol_type = other DNA
aggaggaaaaaaactatg